(19) United States
(12) Patent Application Publication
(10) Pub. No.: US 2025/0205298 A1
(43) Pub. Date: Jun. 26, 2025

(54) HERBAL COMPOSITION FOR ENHANCING SEXUAL WELLNESS AND MUSCLE STRENGTH IN HUMAN BEINGS

(71) Applicants: ANIRUDH MURTHY RANGA, MYSORE (IN); SAMEER KAMALAKAR AKOLKAR, AURANGABAD (IN); MELUKOTE SRINIVASA SHETTY MADHUKUMAR, MYSORE (IN)

(72) Inventors: ANIRUDH MURTHY RANGA, MYSORE (IN); SAMEER KAMALAKAR AKOLKAR, AURANGABAD (IN); MELUKOTE SRINIVASA SHETTY MADHUKUMAR, MYSORE (IN)

(73) Assignee: NATURAL AND ESSENTIAL OILS PRIVATE LIMITED, MYSORE (IN)

(21) Appl. No.: 19/001,337

(22) Filed: Dec. 24, 2024

(30) Foreign Application Priority Data
Dec. 25, 2023 (IN) 202341088601

(51) Int. Cl. A61K 36/48; A61P 15/00

(52) U.S. Cl. CPC A61K 36/48; A61P 15/00

(57) ABSTRACT

The present disclosure envisages a synergistic herbal composition that enhances Testosterone levels in human beings, thus contributing to a consequential improvement in muscle strength, endurance, and muscle recovery. The herbal composition is synthesized from plant parts of a plurality of herbs. The herbal composition comprises a therapeutically effective amount of extracts of a plurality of herbs, including Tamarindus indica, Moringa oleifera, Cocculus hirsutus, Coffea arabica, Psoralea corylifolia, Cinnamon zeylanicum, and Brassica nigra in a predetermined concentration. Further, the extract of Tamarindus indica is standardized to contain polysaccharides and proanthocyanidins in a predetermined concentration and the extract of Moringa oleifera is standardized to contain polysaccharides and saponins in a predetermined concentration. The herbal composition also comprises appropriate biological agents, pharmaceutically acceptable active ingredients, vitamins, minerals, excipients, and carriers and diluents in a predetermined concentration.

### BACKGROUND

The present disclosure relates to the field of herbal compositions. Particularly, the present disclosure relates to herbal compositions that enhance sexual wellness and muscle health in human beings. More particularly, the present disclosure relates to herbal compositions that enhance Testosterone levels in human beings, especially men.

Testosterone is one of the most potent naturally secreted androgenic-anabolic hormones, whose positive biological effects include the promotion of muscle growth and muscle recovery. Testosterone is pivotal in preserving and improving bone health and libido and producing new blood cells. Testosterone is an androgen, which is found in both men and women. Testosterone also plays a vital role in the growth and maintenance of a healthy body. Testosterone is quickly converted into Estrogen in women, while in men, it remains mainly as Testosterone.

In men, Testosterone plays a vital role in body fat distribution, bone density, hair growth-both on the face and body, muscle growth and strength, production of red blood cells, sperm production, mood regulation, motivation, cognitive functions, and sex drive. In women, Testosterone plays a vital role in preserving and enhancing bone health, breast health, fertility, sex drive, menstrual health, mood regulation, cognitive functions, and vaginal health. Typically, Testosterone stimulates protein synthesis—an anabolic effect—and inhibits protein degradation—an anti-catabolic effect. In combination, The anabolic and the anti-catabolic effects promote muscle growth.

Typically, lower levels of Testosterone in men can lead to depression, erectile dysfunction, low sex drive, fatigue, slow muscle growth rate, muscle loss, and slower muscle recovery rates. Further, lower levels of Testosterone in women can lead to depression, fatigue, changes in the breast tissue, low sex drive, osteoporosis, vaginal dryness, and fertility-related issues. Typically, in men, Testosterone activates the intracellular androgen receptor (AR) to regulate gene expressions and cellular functions. Testosterone interacts with the intracellular androgen receptor to regulate male sexual development, muscle growth, and bone density. Testosterone plays a pivotal role in enabling a man's body to adopt to strength training and resistance exercises. Further, Testosterone is considered the major promoter of muscle growth and the subsequent increase in muscle mass and density as a response to strength training and resistance exercises in men.

Testosterone and its levels have invariably been linked to sexual health, sexual productivity, muscle mass, muscle gain, and muscle performance in individuals, especially men. Since it has been established that Testosterone enhances muscle growth, muscle mass, strength, sexual wellness, and athletic performance, a significant amount of research has been undertaken to understand the effects of Testosterone on erectile dysfunction and other related sexual diseases, including fatigue, lack of libido and sexual desire, as well as muscle mass gain, muscle recovery, and enhanced muscle performance.

Further, it has been established that Testosterone promotes the release of nitric oxide in blood vessels, thus facilitating the relaxation of smooth muscles (for example, male genitals) and increasing the blood flow to the smooth muscles. Therefore, based on this premise, a topical system and method for treating sexual dysfunction had been envisaged in U.S. Pat. No. 9,439,926. According to U.S. Pat. No. 9,439,926, a composition containing melanocyte-stimulating hormones (MSH) and molecular nitric oxide was administered topically to treat erectile dysfunction or sexual dysfunction. According to this prior art patent document, the combination of MSH and nitric oxide promoted increased nitric oxide and blood flow to the genitals and thus promoted general feelings of arousal as well as genital arousal. According to U.S. Pat. No. 9,439,926, the combination of MSH and nitric oxide was also used to treat erectile dysfunction by increasing the blood flow to the genitals.

However, prolonged use of MSH and nitric oxide could induce a variety of harmful side effects, including nausea, vomiting, flushing, headache, nasal congestion, dizziness, increased heart rate, and increased blood pressure. Furthermore, nitric oxide is known to have a short half-life, which limits its direct therapeutic application. Further, MSH usually requires to be consumed as a spray, injection, or cream, which can make its application inconvenient. Furthermore, nitric oxide's therapeutic efficacy often depends on precursor medications such as sildenafil or L-arginine, an amino acid. Furthermore, it is well known that excessive use of nitric oxide, in combination with other known vasodilators, can cause hypotension-extremely low blood pressure, which is a life-threatening condition. Furthermore, nitric oxide-based erectile dysfunction treatment is not suitable for people with cardiovascular issues and people who are consuming nitrates in other forms, given that nitric oxide has the propensity to create potentially dangerous interactions when combined with other forms of nitrate. Therefore, in order to obviate the drawbacks or the side effects discussed hitherto, there was felt a need for a herbal composition that enhanced Testosterone levels and exhibited no major side effects, and was safe and convenient to use.

One such effort at envisaging a safe, easy-to-use herbal composition involved the use of Tamarind (Tamarindus indica) and Moringa oleifera to create a synergistic, therapeutically appropriate blend that would promote sexual wellness and improve muscle performance and testosterone levels in human beings.

Tamarind is a commercially valuable dicotyledonous tree grown in Asia, Africa, and America. The various parts of Tamarind, such as the leaves, shell, pulp, fiber, and seeds, find application in the food industry, pharmaceutical industry, biofuel industry, water treatment industry, electrochemical industry, textile industry, and composite industry. The tamarind fruit pulp is used in the food industry as a preservative, while Tamarind Seed Polysaccharide (TSP), a biopolymer from Tamarind seed, is used as an additive in the food industry. Moringa oleifera is a tree belonging to the Moringaceae family, widely cultivated throughout the tropics and subtropics. Moringa oleifera leaves, seed pods, seeds, seed oil, roots, bark, flowers, and sap are commonly used in preparing traditional medicines as well as a source of traditional food.

The present disclosure envisages a synergistic, therapeutically active, and appropriate herbal blend that does not exhibit any of the harmful side effects typically associated with the combination of MSH and nitric oxide, is convenient to use, has a longer shelf-life, and safely enhances the Testosterone levels in human beings, especially men.

An object of the present disclosure is to envisage a therapeutically active and effective herbal composition that enhances Testosterone levels in both men and women.

Yet another object of the present disclosure is to envisage a herbal composition that enhances sexual drive, muscle and bone health, mood, and energy levels in both men and women.

Another object of the present disclosure is to envisage a herbal composition that does not cause any adverse side effects in both men and women.

Yet another object of the present disclosure is to envisage a herbal composition that alleviates medical conditions associated with low Testosterone levels, including loss of libido, erectile dysfunction (ED), female sexual arousal disorder (FSAD), lower energy levels, mood swings, and muscle weakness.

Another object of the present disclosure is to envisage a herbal composition that helps restore energy levels, increase muscle mass, improve mood, and enhance libido in men.

Yet another object of the present disclosure is to envisage a herbal composition that alleviates symptoms associated with low libido in women and is useful in maintaining sexual desire.

One more object of the present disclosure is to envisage a herbal composition that promotes the production of red blood cells, helps with fat distribution in the body, and enhances muscle mass development, strength, mass, and density in men.

Another object of the present disclosure is to envisage a herbal composition that helps prevent bone thinning and Osteoporosis by maintaining bone density.

One more object of the present disclosure is to envisage a herbal composition that regulates fat distribution, promotes fat loss, contributes to a healthy muscle-to-fat ratio, and regulates fat storage.

Yet another object of the present disclosure is to envisage a herbal composition that improves body metabolism in both men and women.

One more object of the present disclosure is to envisage a herbal composition that improves mood, reduces irritability, helps manage symptoms of depression and anxiety, improves focus, memory, and cognitive abilities, improves mental clarity and concentration, and enhances cognitive function and decision-making, in both men and women.

One more object of the present disclosure is to envisage a herbal composition that enhances general vitality and helps maintain energy levels in both men and women.

Another object of the present disclosure is to envisage a herbal composition that enhances Testosterone levels in both men and women and thereby improves the functioning of blood vessels, contributes to better blood circulation, and stimulates the production of blood cells, thereby improving oxygen delivery throughout the body.

Yet another object of the present disclosure is to envisage an herbal composition that enhances Testosterone levels in both men and women, thereby enhancing their body's ability to fight infections and maintain overall health and helping them recover faster from physical exhaustion.

Yet another object of the present disclosure is to envisage a herbal composition that is useful in balancing the Testosterone levels in both men and women, thus contributing to better sexual health, physical appearance, and mental state.

Another object of the present disclosure is to envisage a herbal composition that is useful in reversing erectile dysfunction in men and in enabling men to achieve and maintain an erection.

Another object of the present disclosure is to envisage a herbal composition that enhances Testosterone levels and thus helps men build muscle, achieve greater muscle mass and size, and helps women achieve lean muscle mass.

Another object of the present disclosure is to envisage a herbal composition that enhances Testosterone levels in both men and women and thereby enhances their physical performance in energy-intensive activities such as weightlifting, sports, exercising, and other forms of physical activity.

### SUMMARY

The present disclosure envisages a herbal composition suitable for enhancing sexual wellness and muscle strength in human beings, especially men. The herbal composition envisaged by the present disclosure can be safely consumed also by women to achieve a reasonable enhancement of sexual wellness without any adverse side effects. However, muscle performance-related advantages and sexual well-being-related advantages associated with the use of the herbal composition will be more visible and apparent in men, given that a man's body naturally contains a significantly higher level of Testosterone (300-1000 ng/dL) in comparison to his female counterparts (15-70 ng/dL) and is naturally more receptive and responsive to any therapeutic compositions, such as the herbal composition envisaged by the present disclosure, that aims to enhance testosterone levels.

In accordance with the present disclosure, the herbal composition includes an effective amount of an extract of Tamarindus indica and an effective amount of an extract of Moringa oleifera. In accordance with the present disclosure, the herbal composition comprises the extract of Tamarindus indica in an amount ranging between 10% to 90% by weight of the herbal composition and the extract of Moringa oleifera in an amount ranging between 10% to 90% by weight of the herbal composition.

In accordance with the present disclosure, the herbal composition includes at least one additional extract selected from a group consisting of Cocculus hirsutus extract in an amount up to 35% by weight of the herbal composition, Coffea arabica extract in an amount up to 10% by weight of the herbal composition, Psoralea corylifolia extract in an amount up to 60% by weight of the herbal composition, Cinnamon zeylanicum extract in an amount up to 35% by weight of the herbal composition, and Brassica nigra extract in an amount up to 10% by weight of the herbal composition.

In accordance with the present disclosure, the extract of Tamarindus indica is standardized to contain polysaccharides in a concentration ranging between 0.1% and 60% by weight of the herbal composition and to contain proanthocyanidins in a concentration ranging between 0.1% and 60% by weight of the herbal composition. In accordance with the present disclosure, the extract of Moringa oleifera is standardized to contain polysaccharides with a concentration in a range of 0.1% to 30% by weight of the herbal composition and to contain saponins with a concentration in a range of 0.1% to 50% by weight of the herbal composition.

In accordance with the present disclosure, the herbal composition comprises at least one of fractions, active compounds, phytochemicals, or mixtures thereof derived from plant parts of the Tamarindus indica, Moringa oleifera, Cocculus hirsutus, Coffea arabica, Psoralea corylifolia, Cinnamon zeylanicum, and Brassica nigra.

In accordance with the present disclosure, the herbal composition further comprises extracts of at least one herb selected from a group consisting of Allium cepa, Coffea robusta, Mimosa pudica, Abutilon indicum, Withania somnifera, Tribulus terrestris, and Coleus forskohlii. The herbal composition contains the herb in an amount ranging between 10% and 40% by weight of the herbal composition. The herbal composition also contains one or more of the necessary biological agents, pharmaceutically acceptable active ingredients, vitamins, minerals, excipients, and carriers and diluents, in an amount up to 10% by weight of the herbal composition.

The present disclosure also envisages a method of enhancing sexual wellness and muscle strength in a person, especially a man. The herbal composition envisaged by the present disclosure can be safely consumed also by women to achieve a reasonable enhancement of sexual wellness without any adverse side effects. However, muscle performance-related advantages and sexual well-being-related advantages associated with the use of the herbal composition will be more visible and apparent in men given that a man's body naturally contains a significantly higher level of Testosterone (300-1000 ng/dL) in comparison to his female counterparts (15-70 ng/dL), and is naturally more receptive and responsive to any therapeutic compositions, such as the herbal composition envisaged by the present disclosure, that aims to enhance testosterone levels.

According to the method envisaged by the present disclosure, any person suffering from erectile dysfunction or needing an enhancement in terms of sexual wellness and muscle performance is administered a therapeutically effective dosage of the herbal composition that, in turn, includes an effective amount of an extract of Tamarindus indica and an effective amount of an extract of Moringa oleifera, and at least one additional extract selected from a group consisting of Cocculus hirsutus extract in an amount up to 35% by weight of the herbal composition, Coffea arabica extract in an amount up to 10% by weight of the herbal composition, Psoralea corylifolia extract in an amount up to 60% by weight of the herbal composition, Cinnamon zeylanicum extract in an amount up to 35% by weight of the herbal composition, and Brassica nigra extract in an amount up to 10% by weight of the herbal composition. Preferably, the herbal composition contains the extract of Tamarindus indica in an amount ranging between 10% to 90% by weight of the herbal composition and the extract of Moringa oleifera in an amount ranging between 10% to 90% by weight of the herbal composition.

In accordance with the present disclosure, the herbal composition, when administered over a prescribed period of time in a therapeutically effective quantity, enhances the anabolic and androgenic activities in the human body and manifests improved testosterone levels, improved energy levels, sustained energy levels, sustained vigor, enhanced stamina, healthy aging, cellular longevity, enhanced muscle mass, enhanced muscle strength, enhanced sexual functions, psychological well-being, and reduced stress levels.

Furthermore, the herbal composition, when administered over a prescribed period of time in a therapeutically effective quantity, alleviates symptoms associated with low levels of testosterone in the human body, including loss of libido, loss of erectile function, abdominal obesity, decreased muscle mass and strength, fatigue, mood swings, decreased bone density, decreased motivation, decreased memory, and concentration.

### BRIEF DESCRIPTION OF THE DRAWINGS

FIG. 1 is a clustered column graph illustrating the increase in total Testosterone levels of Sprague Dawley rats during an in-vivo efficacy test conducted to establish the efficacy of the herbal composition envisaged by the present disclosure.

FIG. 2 is a clustered column graph illustrating the increase in free Testosterone levels of Sprague Dawley rats during the in-vivo efficacy test.

FIG. 3 is a clustered column graph illustrating the body weight distribution of human beings (human test subjects) who participated in a placebo-controlled human clinical trial to establish the efficacy of the herbal composition envisaged by the present disclosure.

FIG. 4 is a clustered column graph illustrating the body mass index (BMI) distribution of human beings who participated in the placebo-controlled human clinical trial.

FIG. 5 is a clustered column graph illustrating the increase in total Testosterone levels of human beings who participated in the placebo-controlled human clinical trial.

FIG. 6 is a clustered column graph illustrating the increase in free Testosterone levels of human beings who participated in the placebo-controlled human clinical trial.

FIG. 7 is a clustered column graph illustrating the increase in mTOR levels of human beings who participated in the placebo-controlled human clinical trial.

FIG. 8 is a clustered column graph illustrating the decrease in LDH levels of human beings who participated in the placebo-controlled human clinical trial.

### DETAILED DESCRIPTION

The present disclosure envisages a synergistic herbal composition that, when administered in a therapeutically effective manner and quantity, enhances the anabolic and androgenic activities in humans and manifests improved testosterone levels, improved and sustained energy levels, sustained vigor, enhanced stamina, healthy aging, cellular longevity, enhanced muscle mass, enhanced muscle strength, enhanced sexual functions, psychological well-being, and reduced stress levels.

In accordance with the present disclosure, the herbal composition can be safely consumed, with no side-effects, by both men and women. However, the herbal composition will be comparatively more effective for men and the muscle performance-related advantages and sexual well-being-related advantages envisaged by the present disclosure will be apparent in men given that a man's body naturally contains a significantly higher level of Testosterone (300-1000 ng/dL) in comparison to his female counterparts (15-70 ng/dL), and is likely to be naturally more receptive and responsive to the herbal composition envisaged by the present disclosure that aims to enhance or boost the testosterone levels.

In accordance with the present disclosure, the herbal composition is useful in enhancing testosterone levels in men and women, and more particularly men, and in enabling them to undertake strength and resistance training in an effective and efficient manner. In accordance with the present disclosure, by enhancing Testosterone levels, the herbal composition also helps men—the primary intended consumers of the herbal composition—to recover muscle and build strength in a far more efficient and effective manner.

By enhancing Testosterone levels, the herbal composition envisaged by the present disclosure also promotes muscle gain and strength gain in men, providing them with the necessary stamina and endurance to undertake physically challenging tasks such as working out, jogging, weight lifting, and the like, regularly, without feeling fatigued. For women, the herbal composition envisaged by the present disclosure is useful in treating female sexual arousal disorder (FSAD) and in enhancing sexual performance and experience.

As specified above, the herbal composition envisaged by the present disclosure enhances the testosterone levels in the human body, especially in a man's body. As described hitherto, the herbal composition envisaged by the present disclosure can be safely consumed also by women to achieve a reasonable enhancement of sexual wellness without any adverse side effects.

The herbal composition envisaged by the present disclosure alleviates symptoms associated with low levels of testosterone in a human body, including loss of libido, loss of erectile function (or erectile dysfunction), abdominal obesity, decreased muscle mass and strength, fatigue, mood swings, decreased bone density, decreased motivation, decreased memory, and concentration. By enhancing testosterone levels in the human body, the herbal composition envisaged by the present disclosure reduces and gradually eliminates the symptoms associated with lower levels of Testosterone and reduces the possibility of such symptoms resurfacing/reappearing.

The herbal composition envisaged by the present disclosure is useful in treating the loss of libido in both men and women. Libido, also known as sex drive, is the desire for sexual activity or sexual pleasure. The herbal composition, by enhancing the Testosterone levels, is also useful in enhancing the libido. The herbal composition envisaged by the present disclosure is useful in treating hormone-induced erectile dysfunction (ED) in men and female sexual arousal disorder (FSAD) in women. Erectile dysfunction, typically observed in men, is sexual dysfunction characterized by the inability to develop and maintain an erection of the penis. Female sexual arousal disorder (FSAD), as the name suggests, is a type of female sexual dysfunction characterized by an inability to achieve or maintain sexual arousal. The herbal composition envisaged by the present disclosure is useful in treating both ED and FSAD.

In addition, the herbal composition envisaged by the present disclosure brings about an enhancement in terms of muscle performance and muscle recovery in both men and women, by boosting their Testosterone levels. It is well known that Testosterone promotes muscle protein synthesis, thus aiding in muscle growth and recovery, and improving muscle strength and endurance. It is also well known that Testosterone supports fat metabolism, thus helping both men and women maintain a lean physique. It is also well known that Testosterone boosts energy levels, thus enhancing energy levels and endurance. It is also well known that Testosterone assists in maintaining good bone health, thus reducing the risk of injury during high-impact and high-intensity activities.

By enhancing/boosting Testosterone levels, the herbal composition envisaged by the present disclosure enables both men and women to achieve all the health benefits typically associated with enhanced/increased Testosterone levels, including muscle growth, muscle recovery, enhancement of muscle strength and endurance, enhancement of workout intensity, improved body metabolism, improved fat loss, and improved bone health. The herbal composition envisaged by the present disclosure acts as a catalyst for the enhancement of Testosterone levels and enables consumers/recipients (i.e., people who are administered the herbal composition) to enjoy superior muscle performance and sexual wellness in comparison to those who are affected by lower levels of Testosterone, which, in turn, leads to decreased muscle mass, reduced strength and endurance, and hormone-induced erectile dysfunction, and female sexual arousal disorder inter-alia.

In accordance with the present disclosure, the herbal composition, when consumed in a therapeutically appropriate and effective manner, improves mTOR (Mammalian target of Rapamycin), which, in turn, regulates skeletal muscle mass through the regulation of protein synthesis and degradation pathways in the human body. The herbal composition envisaged by the present disclosure improves mTOR levels in recipients and thus enhances their muscle mass or muscle size.

In accordance with the present disclosure, the herbal composition, when consumed in a therapeutically appropriate and effective manner, significantly increases all the parameters influencing male sexual behavior, including sniffing, mounting, ejaculation frequency, sperm count, mount latency, intromission latency, sperm irregularities, and sperm mobility.

In accordance with the present disclosure, the herbal composition, when consumed in a therapeutically appropriate and effective manner, enhances or improves the production of Luteinizing Hormone (LH), which stimulates the production of Testosterone in men and Progesterone and Estrogen in women. In addition, the herbal composition also enhances or improves Follicle-Stimulating Hormone (FSH), which, in turn, stimulates sperm production and sperm maturation in men and egg development and Estrogen production in women.

In accordance with the present disclosure, the herbal composition, when consumed in a therapeutically appropriate and effective manner, enhances the production of Bone Alkaline Phosphate (Bone ALP) in the human body, which, in turn, plays a vital role in bone formation and bone mineralization (i.e., bone strength) by promoting calcium and phosphate deposition in a recipient's body.

In accordance with the present disclosure, the herbal composition, when consumed in a therapeutically appropriate and effective manner, also enhances the production of Osteocalcin in the human body, which, in turn, plays a vital role in bone metabolism by promoting bone mineralization and maintaining the structural integrity of the bones.

The herbal composition envisaged by the present disclosure is also useful in preventing Osteoporosis in middle-aged men and women and in at least delaying the onset of age-related Osteoporosis in older men and women. Osteoporosis is mainly characterized by reduced bone density and structural deterioration of bone tissues, which, in turn, leads to fragile bones and an increased risk of fractures.

By enhancing the production of Osteocalcin, promoting bone mineralization and bone metabolism, and by helping maintain bone density, the herbal composition envisaged by the present disclosure prevents the onset of Osteoporosis in men and women and at least delays the onset of age-related Osteoporosis in older men and women. The herbal composition envisaged by the present disclosure enhances Testosterone levels in men and Estrogen levels in women. The herbal composition thus helps control Testosterone decline and Estrogen deficiency and reduces the probability of bone loss driven by Testosterone and Estrogen deficiency, which, if unchecked and unhindered, may speed up the onset of Osteoporosis.

In accordance with the present disclosure, the herbal composition is stable and can be stored for prolonged periods of time with little or no loss or reaction of the herbal extracts contained therein. Preferably, the herbal composition is stable at room temperature and could remain active for extended time periods.

In accordance with the present disclosure, the herbal composition can be contained in various forms, including dry powder, semi-solid food, and liquid or beverage. In accordance with the present disclosure, the herbal composition can also be formulated as a food supplement or a dietary supplement. In accordance with the present disclosure, the herbal composition can be formulated into tablets, soft chewable tablets, capsules, and chewing gums inter-alia. In accordance with the present disclosure, the herbal composition can also be formulated as a dietary supplement in the form of chocolates, candy bars, nutritional bars or energy bars, snack bars, and any milk-based desserts inter-alia. Further, the herbal composition can also be formulated as jam, a gel, a yogurt admixture, jelly, or cookies. In addition, the herbal composition envisaged by the present disclosure can also be used an additive to coffee, tea, milk, and select fruit juices. Further, the herbal composition, in the form of a dry powder, can be used as an additive to coffee powder, tea powder, milk powder, and pudding mix inter-alia.

In accordance with the present disclosure, the herbal composition comprises an effective amount of an extract of Tamarindus indica (common name: Tamarind) and an effective amount of an extract of Moringa oleifera (common name: Moringa). Preferably, the herbal composition comprises the extract of Tamarindus indica in an amount ranging between 10% to 90% by weight of the herbal composition. Preferably, the herbal composition comprises the extract of Moringa oleifera in an amount ranging between 10% to 90% by weight of the herbal composition.

In accordance with the present disclosure, the herbal composition further comprises at least one additional herbal extract selected from a group consisting of Cocculus hirsutus (common name: Patalgarudi), Coffea arabica, Psoralea corylifolia (common name: Bakuchi), Cinnamon zeylanicum (common name: Cinnamon), and Brassica nigra (common name: mustard). Preferably, the Cocculus hirsutus extract is present in the herbal composition in an amount up to 35% by weight of the herbal composition. Preferably, the Coffea arabica extract is present in the herbal composition in an amount up to 10% by weight of the herbal composition. Preferably, the Psoralea corylifolia extract is present in the herbal composition in an amount up to 60% by weight of the herbal composition. Preferably, the Cinnamon zeylanicum extract is present in the herbal composition in an amount up to 35% by weight of the herbal composition. Preferably, the Brassica nigra extract is present in the herbal composition in an amount up to 10% by weight of the herbal composition.

In accordance with the present disclosure, the herbal composition further comprises an extract of at least one herb selected from a group consisting of Allium cepa (common name: Onion), Coffea robusta, Mimosa pudica (common name: Lajvanti), Abutilon indicum (common name: Atibala), Withania somnifera (common name: ashwagandha), Tribulus terrestris, and Coleus forskohlii (common name: Indian coleus). In accordance with the present disclosure, the herbal extract selected from the group consisting of Allium cepa, Coffea robusta, Mimosa pudica, Abutilon indicum, Withania somnifera, Tribulus terrestris, and Coleus forskohlii is present in an amount ranging between 10% and 40% by weight of the herbal composition.

In accordance with an exemplary embodiment of the present disclosure, the herbal composition envisaged by the present disclosure further includes an extract of at least one herb selected from the group consisting of Argyreia nervosa (common name: Elephant creeper), Curcuma caesia (common name: Black turmeric), Pueraria tuberosa (common name: Indian kudzu), Jasminum grandiflorum (common name: Jasmine), and Mangifera indica (common name: Mango).

In accordance with the above-mentioned exemplary embodiment of the present disclosure, the herbal extract selected from the group consisting of Argyreia nervosa, Curcuma caesia, Pueraria tuberosa, Jasminum grandiflorum, and Mangifera indica is present in an amount ranging between 9% and 20% by weight of the herbal composition.

In accordance with a preferred embodiment of the present disclosure, the herbal composition comprises the extract of Tamarindus indica, the extract of Moringa oleifera, at least one additional herbal extract selected from the group consisting of Cocculus hirsutus, Coffea arabica, Psoralea corylifolia, Cinnamon zeylanicum, and Brassica nigra, and at least one additional herbal extract selected from the group consisting of Allium cepa, Coffea robusta, Mimosa pudica, Abutilon indicum, Withania somnifera, Tribulus terrestris, and Coleus forskohlii.

In accordance with the present disclosure, instead of the extracts of the above-mentioned herbs, the herbal composition can be synthesized using at least one of fractions, active compounds, phytochemicals, or mixtures thereof derived from the plant parts of the above-mentioned herbs.

In accordance with the present disclosure, the above-mentioned herbs, used for synthesizing the herbal composition are purified on the basis of their molecular weights by ultrafiltration using predetermined ultrafiltration membranes. In accordance with the present disclosure, the process of ultrafiltration involves the use of plant parts of the above-mentioned herbs, with the plant parts being selected from a group consisting of pulp, seeds, leaves, matured stems, tender stems, tender twigs, aerial parts, whole fruits, fruit rinds, roots, bark, and hardwood. Further, it is well within the purview of the present disclosure to use different plant parts of different herbs identified hitherto to synthesize the herbal composition of the present disclosure, with the herbs and the plant parts thereof being selected based on their polymeric forms, molecular weights, and the quantum of extractable bioactive compounds contained therein.

In accordance with another exemplary embodiment of the present disclosure, for the ultrafiltration process, the fruits of Tamarindus Indica can be used in combination with the leaves of Moringa Oleifera, the beans of Coffea arabica, the bark of Cinnamon zeylanicum, the stem and leaves of Cocculus hirsutus, the roots of Withania somnifera, and the stem and leaves of Cocculus hirsutus.

In accordance with yet another exemplary embodiment of the present disclosure, the extracts of Tamarindus indica, Moringa oleifera, Cocculus hirsutus, Coffea arabica, Psoralea corylifolia, Cinnamon zeylanicum, Brassica nigra, Allium cepa, Coffea robusta, Mimosa pudica, Abutilon indicum, Withania somnifera, Tribulus terrestris, and Coleus forskohlii are individually (i.e., separately) subjected to the process of ultrafiltration and the resultant ultrafiltered concentrate is subsequently mixed in a predetermined desired ratio to obtain the herbal composition.

In accordance with the present disclosure, during the preparation of the herbal composition, the extracts of herbs including Tamarindus indica, Moringa oleifera, Cocculus hirsutus, Coffea arabica, Cinnamon zeylanicum, Brassica nigra, Allium cepa, Coffea robusta, Mimosa pudica, Abutilon indicum, Withania somnifera, Tribulus terrestris, and Coleus forskohlii are thoroughly washed and dried to remove any contaminants. As described hitherto, in the preferred embodiment of the present disclosure, each of the above-mentioned herbs are washed and dried separately. It is well within the purview of the present disclosure to make use of fractions, active compounds, phytochemicals, or mixtures thereof in the ultrafiltration process, instead of the extracts.

Subsequently, the extracts of the above-mentioned herbs (i.e., Tamarindus indica, Moringa oleifera, Cocculus hirsutus, Coffea arabica, Cinnamon zeylanicum, Brassica nigra, Allium cepa, Coffea robusta, Mimosa pudica, Abutilon indicum, Withania somnifera, Tribulus terrestris, and Coleus forskohlii) are individually pulverized (or ground) for improving the extraction efficiency. In accordance with the present disclosure, the pulverized extracts of each of the above-mentioned herbs is mixed in a predetermined solvent to extract bioactive compounds such as polysaccharides, proanthocyanidins, and saponins therefrom.

In accordance with the preferred embodiment of the present disclosure, different solvents maintained at different temperature levels and having different compositions (for example, 50% ethanol at 55-60° C., 75% ethanol at 55-60° C., hexane at 55-60° C., and ethyl acetate at 55-60° C.) are used to dissolve different herb extracts. In accordance with the present disclosure, the most preferable solvents are water, ethanol, hexane, ethyl acetate, and methanol. The herb extracts are typically soaked in respective solvents or boiled along with the solvents to extract the bioactive and hydrophilic compounds such as polysaccharides, proanthocyanidins, and saponins.

In accordance with the present disclosure, the solvents dissolve the bioactive compounds based on their polarity. For instance, water is typically used as a solvent for dissolving polysaccharides, and ethanol-water mixture, methanol-water mixture, and ethanol are used as solvents for dissolving proanthocyanidins and saponins.

In accordance with the present disclosure, the extracts soaked or boiled with a solvent are individually filtered using coarse filters to remove large-sized debris. Subsequently, in accordance with the present disclosure, the filtered extracts are individually passed through a Polyethersulfone (PES) membrane, a polyvinylidene fluoride (PVDF) membrane, or a ceramic membrane to filter out low molecular weight phenolics from polysaccharides and similar larger bioactive compounds, including proanthocyanidins and saponins. Preferably, a 5-30 kDa (Kilodalton; a unit of measurement of molecular weight) ultrafiltration membrane is used to filter out or extract low molecular weight phenolics, and a 30-100 kDa ultrafiltration membrane is used to filter out or extract polysaccharides and similar larger bioactive compounds, including proanthocyanidins and saponins. However, it is within the purview of the present disclosure to use any appropriate ultrafiltration membrane depending upon the polymeric forms and molecular weights of the extracts.

In accordance with the present disclosure, during the ultrafiltration process, the temperature is maintained between 25-45° Celsius to ensure the stability of the bioactive compound contained within the extracts. Furthermore, pressure is preferably maintained between 1-5 Bar to optimize the flow of the extracts through the ultrafiltration membrane. Preferably, the soaked or boiled extracts are passed through the ultrafiltration membrane to obtain permeates (filtrates) that contain small-molecule compounds such as sugars and salts, and retentate (concentrates) that is enriched with bioactive components such as polysaccharides, proanthocyanidins, and saponins. In accordance with the present disclosure, the concentrates of the individual herbs—obtained through the ultrafiltration process of individual herb extracts—are mixed in a predetermined, desired ratio to obtain the herbal composition.

In accordance with the preferred embodiment of the present disclosure, the concentrates of the individual herbs are mixed in such a way that the herbal composition comprises Tamarindus indica in an amount ranging between 10% to 90% by weight of the herbal composition and Moringa oleifera in an amount ranging between 10% to 90% by weight of the herbal composition.

In accordance with the preferred embodiment of the present disclosure, the concentrates of the individual herbs are mixed in such a way that the herbal composition comprises-in addition to Tamarindus indica and Moringa oleifera—at least one of Cocculus hirsutus in an amount up to 35% by weight of the herbal composition, Coffea arabica in an amount up to 10% by weight of the herbal composition, Psoralea corylifolia in an amount up to 60% by weight of the herbal composition, Cinnamon zeylanicum in an amount up to 35% by weight of the herbal composition, and Brassica nigra in an amount up to 10% by weight of the herbal composition.

Further, in accordance with the preferred embodiment of the present disclosure, during the ultrafiltration process, the extract of Tamarindus indica is filtered using a suitable ultrafiltration membrane such that that it contains polysaccharides in a concentration ranging between 0.1% and 60% by weight of the herbal composition and proanthocyanidins in a concentration ranging between 0.1% and 60% by weight of the herbal composition.

Further, in accordance with the preferred embodiment of the present disclosure, during the ultrafiltration process, the extract of Moringa oleifera is filtered in such a way that it contains polysaccharides with a concentration in a range of 0.1% to 30% by weight of the herbal composition and saponins in a concentration ranging between 0.1% to 50% by weight of the herbal composition.

In accordance with the present disclosure, the concentrates of individual herbs are mixed together in the predetermined, desired ratio described above. In accordance with the present disclosure, the concentrates of the individual herbs are also filtered through a desired ultrafiltration membrane to achieve the desired concentration of polysaccharides, proanthocyanidins, and saponins described above.

Subsequently, the herbal composition obtained through the ultrafiltration process is spray-dried such that any liquid content contained in the herbal composition is dried-off and the liquid-based or semi-fluid-based herbal composition obtained through the ultrafiltration process is converted into dried powder or granules. Furthermore, the process of spray-drying also protects and preserves the bioactive compounds (polysaccharides, proanthocyanidins, and saponins) contained in the herbal composition.

In accordance with the preferred embodiment of the present disclosure, the herbal composition comprises Tamarindus indica in an amount ranging between 10% to 90% by weight of the herbal composition, Moringa oleifera in an amount ranging between 10% to 90% by weight of the herbal composition, at least one of Cocculus hirsutus in an amount up to 35% by weight of the herbal composition, Coffea arabica in an amount up to 10% by weight of the herbal composition, Psoralea corylifolia in an amount up to 60% by weight of the herbal composition, Cinnamon zeylanicum in an amount up to 35% by weight of the herbal composition, and Brassica nigra in an amount up to 10% by weight of the herbal composition, and at least one of Allium cepa, Coffea robusta, Mimosa pudica, Abutilon indicum, Withania somnifera, Tribulus terrestris, and Coleus forskohlii in an amount ranging between 10% and 40% by weight of the herbal composition.

In accordance with the present disclosure, in addition to the above-mentioned herbal extracts, the herbal composition further includes at least one of biological agents, pharmaceutically acceptable active ingredients, vitamins, minerals, excipients, and carriers and diluents, in an amount up to 10% of the weight of the herbal composition.

In accordance with the present disclosure, the biological agents contained in the herbal composition could include: a) adaptogens that reduce cortisol levels in the human body and enable the human body to better adopt to stress, b) hormone precursors that stimulate the release of luteinizing hormones, which, in turn, enhance the production of Testosterone, c) aromatase inhibitors that reduce the conversion of Testosterone to Estrogen, d) 5-alpha-reductase inhibitors that balance hormone levels and prevent Testosterone from being converted into dihydrotestosterone (DHT), e) antioxidant-rich herbs such as Fenugreek that protect Leydig cells in the genitals from oxidative stress, f) anti-inflammatory agents, g) mineral-rich herbs such as Shilajit that provide essential minerals such as Zinc and Magnesium to the human body, and h) aphrodisiacs that enhance sexual function.

In accordance with the present disclosure, the pharmaceutically acceptable active ingredients contained in the herbal composition could include: a) hormonal modulators that directly increase Testosterone in the endocrine pathway of the human body, b) absorption boosters that enhance the bioavailability of herbal compounds contained in the herbal composition by inhibiting drug-metabolizing enzymes in the liver, c) liposomal carriers and phospholipids that improve the delivery of fat-soluble active ingredients contained in the herbal composition.

In accordance with the present disclosure, the herbal composition could include a combination of Vitamins selected from a group consisting of Vitamin D3, Vitamin B6, Vitamin B12, Vitamin E, Vitamin C, and Vitamin A. It is also within the purview of the present disclosure to include all or only some of the above-mentioned vitamins in various possible combinations in the herbal composition. In accordance with the present disclosure, the above-mentioned vitamins, when combined with the herbal composition envisaged by the present disclosure create a supportive environment for Testosterone synthesis by a) directly enhancing Testosterone-related processes, b) balancing cortisol, estrogen, and prolactin levels, and c) preventing oxidative damage that impairs Testosterone production.

In accordance with the present disclosure, the herbal composition could include at least one mineral selected from the group consisting of Zinc, Magnesium, Selenium, Boron, Chromium, Iron, Calcium, and Copper. It is also within the purview of the present disclosure to include all or only some of the above-mentioned minerals in various possible combinations in the herbal composition. In accordance with the present disclosure, the above-mentioned minerals, when combined with the herbal composition envisaged by the present disclosure, ensure a) direct stimulation of Testosterone synthesis, b) absorption of active compounds, c) reduction in oxidative stress and inflammation in the human body, and d) insulin regulation and reduction in stress-related hormone imbalances.

In accordance with the present disclosure, the herbal composition includes excipients that, even though pharmacologically inactive, significantly influence the herbal composition's efficacy by ensuring the herbal composition's stability, bioavailability, and consistent performance. In accordance with the present disclosure, the excipients a) improve the absorption of the active herbal ingredients contained in the herbal composition by enhancing their solubility and reducing their degradation in the digestive tract, b) prevent the oxidation of active herbal components contained in the herbal composition and ensure the stability and shelf-life of the herbal composition, c) hold the herbal components contained in the herbal composition together when the herbal composition is formulated as tablets or capsules, d) facilitate the breakdown of tablets or capsules for faster release of active ingredients contained in the herbal composition, e) protect active ingredients contained in the herbal composition from environmental factors such as moisture and light, f) prevent microbial contamination when the herbal composition is formulated as a liquid or semi-solid, g) aid in the dispersion of herbal compounds contained in the herbal composition, and h) maintain an optimal pH environment for maximum stability and effectiveness of the herbal ingredients contained in the herbal composition. In accordance with the present disclosure, the excipients are selected from a group consisting of monosaccharides, disaccharides, polycarbonates, and modified starch.

In accordance with the present disclosure, the herbal composition includes carriers and diluents that deliver and/or hold active herbal components contained in the herbal composition to ensure their proper dispersion, absorption, and release. In accordance with the present disclosure, the carriers a) enhance the solubility of bioactive materials such as saponins and increase their absorption in the digestive tract, b) allow for sustained release of the active herbal components contained in the herbal composition, c) protect sensitive herbal compounds contained in the herbal composition from oxidation, moisture, and heat, d) deliver active compounds directly to target tissues and thereby improve the efficacy of the herbal composition and reduce the side effects, if any.

In accordance with the present disclosure, diluents are added to the herbal composition envisaged by the present disclosure to bulk up the formulation for appropriate dosing and improved product handling. In accordance with the present disclosure, the diluents are added to the herbal composition to a) add volume to any small doses of active ingredients contained within the herbal composition, b) distribute the active ingredients evenly throughout the herbal composition formulation, thereby ensuring uniform capsule fill or uniform tablet size, c) absorb moisture, prevent clumping, and increase the shelf-life and stability of the herbal composition, d) prevent sticking or clogging during manufacturing, thereby ensuring smooth production, and e) improve tablet compressibility and mechanical strength (in the event the herbal composition is formulated as a tablet) and prevent breakage during transport and handling.

In accordance with the present disclosure, the carriers and diluents are selected from a group consisting of polyhydric alcohols, sugar alcohols, cellulose-based derivatives, silicates, metallic stearates, organic acids, fatty acid esters, esters of polysorbate, Vitamin B, nicotinamide, calcium pantothenate, amino acids, proteins, and organic metal salts.

The present disclosure also envisages a method of administering the herbal composition discussed herein to a subject (i.e., human beings, preferably men). In accordance with the present disclosure, the method includes the step of administering to the subject the herbal composition, which, in turn, comprises Tamarindus indica in an amount ranging between 10% to 90% by weight of the herbal composition, Moringa oleifera in an amount ranging between 10% to 90% by weight of the herbal composition, at least one of Cocculus hirsutus in an amount up to 35% by weight of the herbal composition, Coffea arabica in an amount up to 10% by weight of the herbal composition, Psoralea corylifolia in an amount up to 60% by weight of the herbal composition, Cinnamon zeylanicum in an amount up to 35% by weight of the herbal composition, and Brassica nigra in an amount up to 10% by weight of the herbal composition, and at least one of Allium cepa, Coffea robusta, Mimosa pudica, Abutilon indicum, Withania somnifera, Tribulus terrestris, and Coleus forskohlii in an amount ranging between 10% and 40% by weight of the herbal composition.

The herbal composition envisaged by the present disclosure is administered in a therapeutically effective, pharmaceutically acceptable amount. The herbal composition envisaged by the present disclosure can be administered to both men and women. When administered, the herbal composition does not exhibit any adverse side effects in both men and women.

Preferably, the herbal composition envisaged by the present disclosure is administered to the subject in a therapeutically effective dose. The effective amounts of the herbal composition to be administered to the subject typically depend on the desired outcome. In accordance with the present disclosure, the administration of the herbal composition is designed to cause sequential exposures to the composition over a certain time period, for example, hours, days, weeks, months, or years. This is accomplished by repeated administrations of the herbal composition envisaged by the present disclosure.

As described hitherto, the herbal composition envisaged by the present disclosure is used for treating sexual dysfunction in men and women, i.e., ED in men and FSAD in women. In accordance with the present disclosure, gradual improvement in terms of curative/therapeutic effects is observed with successive administration of the herbal composition over a predetermined period of time.

When administered to men, the herbal composition envisaged by the present disclosure brings about a reasonable enhancement of Testosterone levels and thus promotes improved body fat distribution, improved bone density, improved hair growth-both on the face and body, improved muscle growth and strength, enhanced production of red blood cells, enhanced sperm production, improved mood regulation, improved motivation, improved cognitive functions, and improved sex drive.

When administered to women, the herbal composition envisaged by the present disclosure brings about a reasonable enhancement of Testosterone levels and thus facilitates preservation and enhancement of bone health, breast health, fertility, sex drive, menstrual health, mood regulation, cognitive functions, and vaginal health.

In accordance with the present disclosure, the herbal composition, when administered to men and women in a therapeutically effective manner, enhances anabolic and androgenic activities and provides the recipients with at least one health benefit selected from a group consisting of improved testosterone levels, improved energy levels, sustained energy levels, sustained vigor, enhanced stamina, healthy aging, cellular longevity, enhanced muscle mass, enhanced muscle strength, enhanced sexual functions, psychological well-being, and reduced stress levels.

In accordance with the present disclosure, the herbal composition, when administered to men and women in a therapeutically effective manner, alleviates symptoms associated with low levels of testosterone in recipients' bodies, including loss of libido, loss of erectile function, abdominal obesity, decreased muscle mass and strength, fatigue, mood swings, decreased bone density, decreased motivation, decreased memory, and concentration.

In accordance with the present disclosure, the herbal composition, when administered to a subject (i.e., a human being, preferably a man) in a therapeutically effective manner and as a therapeutically effective dose, controls the secretion/production of two key enzymes, namely Phosphodiesterase-5 (PDE-5) and Aromatase. In accordance with the present disclosure, the PDE-5 enzyme influences blood flow, erectile function, and hormonal balance in the human body. Typically, PDE-5 breaks down cyclic guanosine monophosphate (CGMP), which, in turn, regulates blood vessel relaxation.

The herbal composition of the present disclosure regulates the production of PDE-5, prevents the breakdown of the cGMP, and therefore enhances cGMP levels in the human body, thus promoting vasodilation and improving blood flow to the male genitals (testes), which are responsible for producing Testosterone. Further, in accordance with the present disclosure, better testicular blood flow enhances the delivery of oxygen and nutrients to the testes and supports Leydig cell function, due to which Testosterone is produced.

Further, by regulating the production of PDE-5, the herbal composition of present disclosure improves vascular health, which, in turn, optimizes the pituitary function and stimulates the secretion of Luteinizing Hormones (LH), with the increase in LH secretion stimulating the male genitals (testes) to produce more Testosterone. Further, by regulating the production of PDE-5, the herbal composition of the present disclosure also reduces oxidative stress in the testes and other tissues, thereby lowering the oxidative damage to Leydig cells, protecting the Leydig cells, and preserving the Leydig cells' ability to produce Testosterone efficiently.

Further, by regulating the production of PDE-5, the herbal composition of the present disclosure also improves erectile function, which, in turn, triggers the hypothalamic-pituitary-gonadal axis and stimulates Testosterone production. Further, by regulating the production of PDE-5, the herbal composition of the present disclosure enhances nitric oxide (NO) production, thereby improving vascular function and lowering cortisol levels. In accordance with the present disclosure, lower cortisol levels help maintain a favorable testosterone-to-cortisol ratio, thereby supporting anabolic processes, including the production of Testosterone.

Further, the herbal composition envisaged by the present disclosure also elevates the mechanistic target of rapamycin (mTOR) levels. Further, Testosterone directly activates the mTOR signaling pathway by binding to androgen receptors in the human body. Further, the activation of the mTOR signaling pathway stimulates muscle protein synthesis and cellular growth while inhibiting autophagy and reducing muscle breakdown, thereby promoting an increase in muscle fiber size.

Further, by controlling the production of Aromatase, the herbal composition envisaged by the present disclosure ensures appropriate aromatase activity and ensures a balanced testosterone-to-estrogen ratio, which is essential for male reproductive health, libido, and muscle growth. By regulating/controlling Aromatase production, the herbal composition envisaged by the present disclosure controls the Estrogen levels in the human body and helps maintain Testosterone production and the Estrogen-to-Testosterone ratio.

Further, by controlling Aromatase production, the herbal composition envisaged by the present disclosure optimizes Testosterone production and controls fat accumulation (gynecomastia). Further, by regulating Aromatase production, the herbal composition envisaged by the present disclosure maintains Testosterone production at an optimal level and enhances muscle development. Further, by controlling Aromatase production and the consequential Estrogen secretion, the herbal composition envisaged by the present disclosure enhances libido, cognition, and mood regulation. Further, by balancing Estrogen levels, the herbal composition envisaged by the present disclosure ensures optimum brain function and boosts Testosterone-driven behaviors such as motivation and sexual drive.

In accordance with the present disclosure, an in-vivo efficacy test was performed on three equally distributed groups of Sprague Dawley rats to determine the efficacy of the herbal composition in terms of enhancing Testosterone levels and thus contributing to improved sexual wellness. The three equally distributed groups of Sprague Dawley rats included male rats and female rats in equal numbers. Each of the three groups contained sixteen rats—amongst the sixteen rats, eight rats were male, and the remaining eight were female. The herbal composition envisaged by the present disclosure was orally administered to all the rats in each of the three groups for a time period of fifty consecutive days. The dosage of the herbal composition was determined based on the body weight of the individual rats.

Further, during the in-vivo efficacy test, a first group of rats was categorized as a normal control group that did not receive the herbal composition of the present disclosure. Further, a second group of rats were administered a low dose (i.e., 41.13 milligrams (mg) per kilogram (kg) of body weight) of the herbal composition envisaged by the present disclosure for the time period of fifty consecutive days and a third group of rats were administered a high dose (i.e., 82.26 milligrams (mg) per kilogram (kg) of body weight) of the herbal composition envisaged by the present disclosure for the same time period.

The results achieved by the in-vivo efficacy test demonstrated a marked increase in the total Testosterone levels and free Testosterone levels of the rats that were administered the herbal composition in dosages of 41.13 mg/kg and 82.26 mg/kg, respectively, for the time period of fifty consecutive days, in comparison to the total Testosterone levels and free Testosterone levels of the rats categorized as the normal control group.

Further, the results achieved by the in-vivo efficacy test, after the herbal composition was administered for fifty consecutive days to the rats of the second group and third group, also demonstrated a marked enhancement of the sexual behavior-related parameters in rats belonging to the second and third groups, including mounting latency, intromission latency, and ejaculatory latency.

In accordance with the present disclosure, Table 1, provided below, illustrates the total Testosterone levels observed in rats. The rats belonging to the first group were not administered the herbal composition, and the rats belonging to the second group and third group were administered 41.13 mg/kg and 82.26 mg/kg doses of the herbal composition for a consecutive time period of fifty days starting from day 0.

In accordance with the present disclosure, Table 1 indicates the total Testosterone levels observed on day 0, day 25, and day 50 of the in-vivo efficacy test in the three groups of rats.

TABLE 1
Total Testosterone Levels observed in Sprague Dawley rats

| Group | Total Testosterone Levels (ng/mL) | | | |
|---|---|---|---|---|
| No. | Group Type | Day 0 | Day 25 | Day 50 |
| Group 1 | Normal control | 22.77 ± 2.60 | 25.88 ± 2.05 | 32.66 ± 3.74 |
| | group | | | |
| Group 2 | Group with the | 22.40 ± 2.53 | 30.84 ± 2.71 | 45.64 ± 2.99 |
| | dosage of 41.13 | | (p-value < | (p-value < |
| | mg/kg | | 0.001) | 0.0001) |
| Group 3 | Group with the | 23.57 ± 1.64 | 32.46 ± 2.92 | 50.66 ± 8.13 |
| | dosage of 82.26 | | (p-value < | (p-value < |
| | mg/kg | | 0.0001) | 0.0001) |

In accordance with the present disclosure, the total Testosterone level-related values described in Table 1 for days 0, 25, and 50 were calculated using the expression ‘mean±SD’, with the sample size (n) being equivalent to eight (i.e., sample size derived based on the total number male rats and female rats in every group). Typically, the expression ‘mean±standard deviation (mean±SD)’ depicts the central value (the mean) along with a measure of the spread or variability (the standard deviation).

Further, the values of ‘mean±SD’ of the first group (of rats) were statistically compared with the ‘mean±SD’ values of the second group and third group (of rats) for day 25 and day 50. In accordance with the present disclosure, the ‘mean±SD’ values of the second group and third group (of rats) showed a significant increase in terms of total Testosterone levels vis-à-vis the first group of rats that was not administered the herbal composition.

Further, in any statistical analysis, p-value suggests strong evidence against the null hypothesis and provides for the null hypothesis to be rejected in favor of the alternative hypothesis derived by the statistical analysis. Further, when the efficacy and efficiency of the herbal composition of the present disclosure is being tested through the in-vivo test, the ‘null hypothesis’ is that the “herbal composition has no effect.”

As illustrated in Table 1, the p-value obtained for the total Testosterone levels of rats belonging to group 2 on ‘day 25’ is less than 0.001, and ‘day 50’ is less than 0.0001. Further, as shown in Table 1, the p-value obtained for total Testosterone levels of rats belonging to group 3 on ‘day 25’ as well as ‘day 50’ is less than 0.0001. The typical, well-known threshold value for statistically determining the significance of readings (illustrated in Table 1) is 0.05. Typically, a p-value less than the threshold value of 0.05 implies that there is less than a five-percent chance that the readings (described in Table 1) occurred under the null hypothesis and were a resultant of a random variation.

Further, since the p-values for total Testosterone levels of the rats belonging to group 2 and group 3, for days 25 and 50, are lesser than the threshold value of 0.05, the total Testosterone readings/values depicted in Table 1 demonstrate the effectiveness and efficacy of the herbal composition envisaged by the present disclosure in enhancing the Testosterone levels in Sprague Dawley rats and thus form the basis for the rejection of the null hypothesis, and indicate that the possibility of the null hypothesis being true, in this case, would not exceed five percent.

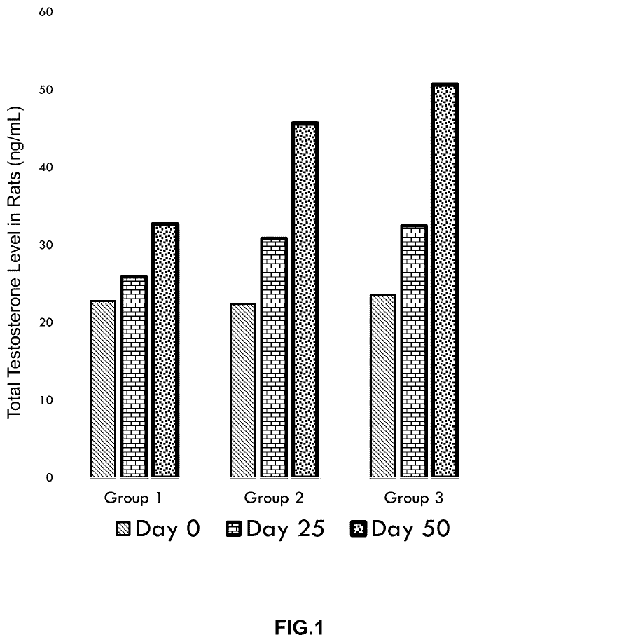

In accordance with the present disclosure, FIG. 1 illustrates a clustered column graph that describes the increase in the total Testosterone levels of Sprague Dawley rats on days 0, 25, and 50 of the in-vivo efficacy test. In FIG. 1, the increase in the total Testosterone levels is plotted on the vertical axis (y-axis), and the days on which the Testosterone level readings were taken are indicated on the horizontal axis (x-axis). In accordance with the present disclosure, FIG. 1 is a graphical representation of the total Testosterone level-related readings illustrated in Table 1.

In accordance with the present disclosure, Table 2, provided below, illustrates the free Testosterone level-related values observed in rats. The rats belonging to the first group were not administered the herbal composition, and the rats belonging to the second group and third groups were administered 41.13 mg/kg and 82.26 mg/kg doses of the herbal composition, respectively, for a consecutive time period of fifty days, starting from ‘day 0’. In accordance with the present disclosure, Table 2 indicates the free Testosterone levels observed on day 0, day 25, and day 50 of the in-vivo efficacy test in the three groups of rats.

The free Testosterone level-related values described in Table 2 for days 0, 25, and 50 were calculated using the expression ‘mean±SD,’ with the sample size (n) being equivalent to eight (i.e., sample size derived based on the total number male rats and female rats present in every group). The values of ‘mean±SD’ of the first group (of rats) were statistically compared with the ‘mean±SD’ values of the second group and third group (of rats) for day 25 and day 50, and the ‘mean±SD’ values of the second group and third group (of rats) showed a significant increase in terms of free Testosterone levels vis-à-vis the first group of rats that was not administered the herbal composition.

TABLE 2
Free Testosterone Levels observed in the Sprague Dawley rats

| Group | Free Testosterone Levels (ng/L) | | | |
|---|---|---|---|---|
| No. | Group Type | Day 0 | Day 25 | Day 50 |
| Group 1 | Normal control | 90.02 ± | 106.85 ± | 132.46 ± |
| | group | 13.02 | 15.78 | 7.83 |
| Group 2 | Group with the | 90.24 ± | 134.07 ± | 165.37 ± |
| | dosage of | 9.02 | 19.70 | 11.81 |
| | 41.13 mg/kg | | (p-value < | (p-value < |
| | | | 0.001) | 0.0001) |
| Group 3 | Group with the | 95.38 ± | 140.98 ± | 171.63 ± |
| | dosage of | 11.92 | 13.22 | 8.51 |
| | 82.26 mg/kg | | (p-value < | (p-value < |
| | | | 0.0001) | 0.0001) |

As shown in Table 2, the p-value obtained for the free Testosterone levels of rats belonging to group 2 on ‘day 25’ is less than 0.001, and ‘day 50’ is less than 0.0001. Further, as shown in Table 2, the p-value obtained for free Testosterone levels of rats belonging to group 3 on ‘day 25’ as well as ‘day 50’ is less than 0.0001.

Further, since the p-values for free Testosterone levels of the rats belonging to group 2 and group 3, for day 25 and day 50, are lesser than the threshold value of 0.05, the free Testosterone readings/values depicted in Table 2 demonstrate the effectiveness and efficacy of the herbal composition envisaged by the present disclosure in enhancing the Testosterone levels in Sprague Dawley rats and thus form the basis for the rejection of the null hypothesis and indicate that the possibility of the null hypothesis being true, in this case, would not exceed five percent.

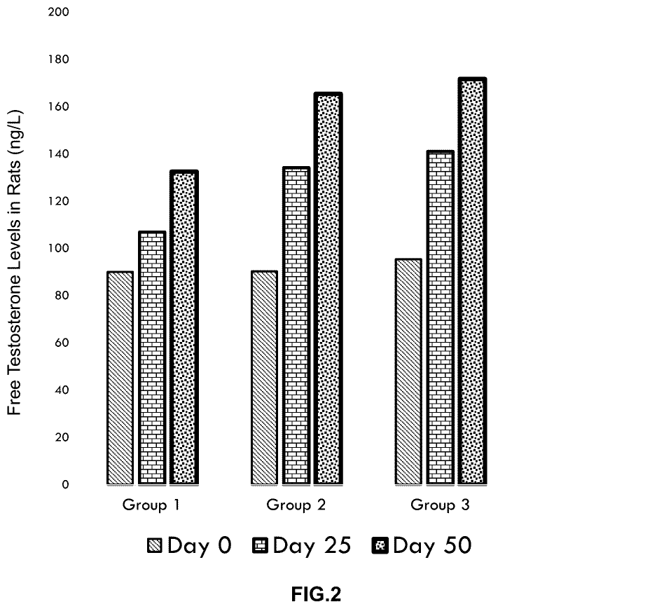

In accordance with the present disclosure, FIG. 2 illustrates a clustered column graph that describes the increase in the free Testosterone levels of Sprague Dawley rats on days 0, 25, and 50 of the in-vivo efficacy test. In FIG. 2, the increase in the free Testosterone levels is plotted on the vertical axis (y-axis), and the days on which the free Testosterone level readings were taken are indicated on the horizontal axis (x-axis). In accordance with the present disclosure, FIG. 2 is a graphical representation of the total Testosterone level-related readings illustrated in Table 2.

In accordance with the present disclosure, during the in-vivo efficacy test, the sexual behavior of Sprague Dawley rats was analyzed in terms of mount latency, intromission, ejaculation, mount percentage, and intromission latency. Typically, intromission latency, measured in seconds, is the time between when a receptive female rat is introduced into an arena and the male rat achieves vaginal penetration with the female rat. Typically, the mount latency, also measured in seconds, is the time taken between when a female rat is introduced into an arena and a male rat first mounts the female rat. Further, intromission is typically a sexual behavior that involves a male rat's copulatory organ (genitals) being inserted into a female rat's genital orifice (vagina).

Typically, the intromission latency, mount latency, intromission, ejaculation, and mount percentage are used to determine a male rat's sexual motivation. These parameters function as the biomarkers of sexual drive, desire, and libido. Typically, a short latency to mount and a short latency to achieve vaginal penetration indicates that a male rat is sexually motivated. Likewise, higher intromission, ejaculation, and mount percentage also indicate that a male rat is sexually motivated.

In accordance with the present disclosure, Table 3, provided below, illustrates the readings observed on ‘day 0’ in respect of the sexual behavior (i.e., intromission latency, mount latency, intromission, ejaculation, and mount percentage) observed in rats during the in-vivo efficacy study.

In accordance with the present disclosure, the rats belonging to the first group were not administered the herbal composition, and the rats belonging to the second group and third group were administered 41.13 mg/kg and 82.26 mg/kg doses of the herbal composition, respectively, beginning from ‘day 0’ through to ‘day 50’. In accordance with the present disclosure, all the sexual behavior-related values illustrated in Table 3 and Table 4, for days 0 and 50, respectively, were calculated using the expression ‘mean±SD,’ with the sample size (n) being equivalent to eight (i.e. sample size derived based on the total number of male rats and female rats in every group).

In accordance with the present disclosure, the sexual behavior-related values observed on ‘day 0’ for the three groups of rats were compared with the sexual behavior-related values observed on ‘day 50’ for the three groups of rats. In accordance with the present disclosure, the second group and third group (of rats) that were administered the herbal composition in doses of 41.13 mg/kg and 82.26 mg/kg, respectively, showed a significant improvement in sexual behavior on ‘day 50’ in relation to ‘day 0’. Also, the second group and third group (of rats) that were administered with the herbal composition showed a significant improvement in terms of sexual behavior (on day 50) vis-à-vis the first group of rats that were not administered the herbal composition.

TABLE 3
Effect of the herbal composition on sexual behavior of rats on ‘day 0’

| | Sexual Behavior observed in rats on Day 0 | | | | |
|---|---|---|---|---|---|
| Group | | No. of | Ejacu- | Mount | Intromission |
| No. and | Mount | Intro- | lation | Latency | Latency |
| Type | (%) | mission | (%) | (%) | (Seconds) |
| Group 1 | 57.08 ± | 8.38 ± | 5.50 ± | 179.63 ± | 226.33 ± |
| Normal | 11.49 | 2.07 | 1.20 | 8.53 | 11.96 |
| control | | | | | |
| group | | | | | |
| Group 2 | 59.85 ± | 7.13 ± | 5.63 ± | 166.75 ± | 221.25 ± |
| Dosage | 11.64 | 1.55 | 1.60 | 22.52 | 15.31 |
| of 41.13 | | | | | |
| mg/kg | | | | | |
| Group 3 | 52.07 ± | 7.38 ± | 5.75 ± | 162.88 ± | 218.25 ± |
| Dosage | 11.33 | 1.41 | 1.39 | 23.81 | 15.31 |
| of 82.26 | | | | | |
| mg/kg | | | | | |

In accordance with the present disclosure, Table 4, provided below, illustrates the readings observed on ‘day 50’ with respect to the sexual behavior observed in rats.

TABLE 4
Effect of the herbal composition on sexual behavior of rats on day 50

| | Sexual Behavior observed in rats on Day 50 | | | | |
|---|---|---|---|---|---|
| Group | | No. of | Ejacu- | Mount | Intromission |
| No. and | Mount | Intro- | lation | Latency | Latency |
| Type | (%) | mission | (%) | (%) | (Seconds) |
| Group 1 | 51.64 ± | 7.00 ± | 6.00 ± | 169.13 ± | 222.13 ± |
| Normal | 15.14 | 1.51 | 2.00 | 14.58 | 18.89 |
| control | | | | | |
| group | | | | | |
| Group 2 | 69.97 ± | 10.75 ± | 10.50 ± | 133.25 ± | 156.25 ± |
| Dosage | 13.23 | 1.98 | 3.16 | 22.52 | 7.67 |
| of 41.13 | (p-value < | (p-value < | (p-value < | (p-value < | (p-value < |
| mg/kg | 0.05) | 0.0001) | 0.001) | 0.05) | 0.0001) |
| Group 3 | 83.38 ± | 13.50 ± | 13.75 ± | 104.75 ± | 113.13 ± |
| Dosage | 6.21 | 1.60 | 2.60 | 29.53 | 8.66 |
| of 82.26 | (p-value < | (p-value < | (p-value < | (p-value < | (p-value < |
| mg/kg | 0.0001) | 0.0001) | 0.0001) | 0.0001) | 0.0001) |

As illustrated in Table 4, the mount percentage, number of intromissions, and ejaculation percentage observed in rats belonging to the second group and third group of rats on ‘day 50’ were significantly higher than the mount percentage, number of intromissions, and ejaculation percentage observed in the second and third groups of rats on ‘day 0’. Likewise, the mount latency and intromission latency observed in rats belonging to the second group and third group on ‘day 50’ were significantly lesser than the mount latency and intromission latency observed in the second and third groups of rats on ‘day 0’.

In accordance with the present disclosure, the mount percentage, number of intromissions, and ejaculation percentage observed in rats belonging to the second group and third group on ‘day 50’ were significantly higher than the mount percentage, number of intromissions, and ejaculation percentage observed on ‘day 0’ and ‘day 50’ in rats belonging to the first group (of rats) that was not administered the herbal composition. Likewise, the mount latency and intromission latency observed in rats belonging to the second group and third group on ‘day 50’ were significantly lesser than the mount latency and intromission latency observed in the first group of rats (that were not administered the herbal composition) on ‘day 0’ as well as ‘day 50’.

Further, as illustrated in Table 4, the p-values for mount percentage, number of intromissions, ejaculation percentage, mount latency, and intromission latency observed on the rats belonging to group 2 and group 3 for day 50 are significantly lesser than the threshold value of 0.05. Therefore, the values related to mount percentage, number of intromissions, ejaculation percentage, mount latency, and intromission latency, as illustrated in Table 4, demonstrate the effectiveness and efficacy of the herbal composition envisaged by the present disclosure in terms of enhancing the sexual behavior of rats and thus form the basis for the rejection of the null hypothesis, and also indicate that the possibility of the null hypothesis being true, in this case, would not exceed five percent.

As per the sexual behavior-related readings/values illustrated in Table 4, the herbal composition envisaged by the present disclosure significantly enhances the mount percentage, number of intromissions, and ejaculation percentage in rats while bringing about a significant decrease in the mount latency and intromission latency attributed to the rats. In accordance with the present disclosure, an increase in the mount percentage, number of intromissions, and ejaculation percentage, as well as a decrease in the mount latency and intromission latency, indicate an improved sexual behavior in rats.

Therefore, from the readings illustrated in Tables 1-4, it is apparent that the herbal composition envisaged by the present disclosure brings about a remarkable and significant improvement in the total Testosterone levels, free Testosterone levels and sexual behavior of rats, thus affirming the efficacy of the herbal composition in enhancing the Testosterone levels in rats and the sexual behavior of the rats.

In accordance with the present disclosure, a placebo-controlled clinical trial involving human beings as the test subjects was also conducted to further establish the efficacy and safety of the herbal composition in improving muscle strength and endurance by enhancing Testosterone levels. The clinical trial entailed the participation of forty-eight healthy male subjects who were made to undergo a training regimen consisting of four resistance workout sessions per week, each workout session lasting at least an hour. The subjects were divided into a treatment group and a placebo group. Preferably, the subjects in the treatment group were administered a capsule containing 500 milligrams of the herbal composition every day for sixty consecutive days, while the placebo group did not receive the herbal composition. The age of the subjects placed in the treatment group was averaged at thirty-one, and the age of the subjects placed in the placebo group was averaged at twenty-four. Both the treatment group and placebo group contained twenty-four participants each.

In accordance with the present disclosure, the male subjects belonging to both the groups were made to perform a variety of resistance exercises, including exercises for the chest, shoulder, back, leg, and arms. During the resistance workout sessions, the subjects were made to perform warm-up exercises at fifty percent of their one-rep max (1RM) capacity for eight to ten repetitions of two sets. Typically, one-rep max (1RM) refers to the maximum amount of weight a person can lift for a given exercise, specifically in one single repetition. Typically, one-rep max is used as a benchmark for measuring the strength of an individual in resistance training, especially in exercises like the bench press (chest exercise), squat (leg exercise), and deadlift (back exercise). Subsequently, the subjects were made to perform two to three sets of repetitions of the above-mentioned exercises at seventy percent of baseline 1 RM. The subjects were allowed a two-minute rest between sets of exercises. Further, the subjects were made to perform two to three sets of repetitions of the above-mentioned exercises at ninety percent of baseline 1 RM, with eight to ten repetitions until failure. Further, the subjects were allowed a ten-minute rest window between two different exercises.

While the subjects performed the exercises, their 1RM strength, handgrip strength, muscle endurance, time to exhaustion, and mid-upper arm circumference (MUAC) were measured. In accordance with the present disclosure, during the assessment of the 1RM strength, the subjects performed eight to ten repetitions of the above-mentioned exercises at approximately fifty percent of their anticipated maximum possible weight, followed by successive lifts starting at seventy percent of their anticipated 1RM. Subsequently, the subjects increased the weights by five kilograms per set until they reached their respective 1RM.

In accordance with the present disclosure, a pre-calibrated analog grip dynamometer was used to measure the handgrip strength of the subjects' dominant hands when they squeezed the dynamometer for at least three seconds, in a standing position, keeping their shoulders adducted, arms by their side with full elbow extension.

In accordance with the present disclosure, the muscle endurance of the subjects was measured using a standardized lateral pull-down cable system. The subjects were made to perform a wide-grip anterior pull-down exercise with their pronated handgrips using a standardized lateral pull-down bar. Preferably, muscle endurance was measured as the number of repetitions the subjects completed at eighty percent of their 1RM by cable pull-down. The subjects performed each pull with completely extended arms until the bar contacted their chest.

Further, in accordance with the present disclosure, the subjects' time to exhaustion was measured by making them run on a treadmill at a speed of six kilometers per hour and zero inclination for three minutes, followed by a two percent increase in the inclination of the treadmill every two minutes until the subjects were no longer able to run.

Further, the time spent by each of the subjects on the treadmill and the treadmill inclinations at various points in time were recorded and analysed to determine the time spent by the subjects on the treadmill until exhaustion. Further, the mid-upper arm circumference (MUAC) of each of the subjects was measured by placing a centimetre-scale measuring tape around the flexed biceps of each of the subjects at the midpoint between their shoulder and the tip of their elbow.

In accordance with the present disclosure, Table 5, provided below, illustrates the body weight of each of the subjects in both the treatment group and placebo group during the time period (sixty days) when the clinical trial was conducted.

TABLE 5
Body weight measurements of the clinical trial subjects

| All the values | Body Weight (Kilograms) | |
|---|---|---|
| represented as | Herbal Composition | |
| ‘mean ± SD’ | (Treatment Group) | Placebo Group |
| Screening | 76.05 ± 11.17 | 74.04 ± 9.26 |
| Day 0 | 76.05 ± 11.17 | 74.04 ± 9.26 |
| Day 15 | 76.20 ± 11.07 | 74.20 ± 9.31 |
| Day 30 | 76.54 ± 11.01 | 74.45 ± 9.26 |
| Day 60 | 77.06 ± 10.91 | 74.79 ± 9.23 |

In accordance with the present disclosure, Table 5 illustrates the body weight variations across a time period of sixty days in each of the subjects who participated in the clinical trial. All the values in Table 5 are represented using the expression ‘mean±SD’, with the sample size (n) considered equivalent to twenty-four (i.e., sample size derived based on the total number of participants in the treatment group and placebo group). Typically, the expression ‘mean±standard deviation (mean±SD)’ depicts the central value (the mean) along with a measure of the spread or variability (the standard deviation).

From the body weight-related values tabulated in Table 5, it is apparent that the herbal composition did not cause any weight gain, rapid or otherwise, in the subjects who were administered the herbal composition daily for a period of sixty days and that the body weight of the subjects showed an extremely low, insignificant variation for the time period of sixty days during which the herbal composition was administered to them on a daily basis.

Therefore, from Table 5, it can be inferred that the herbal composition envisaged by the present disclosure does not cause any adverse weight gain in consumers/recipients when administered for a time period of at least sixty days.

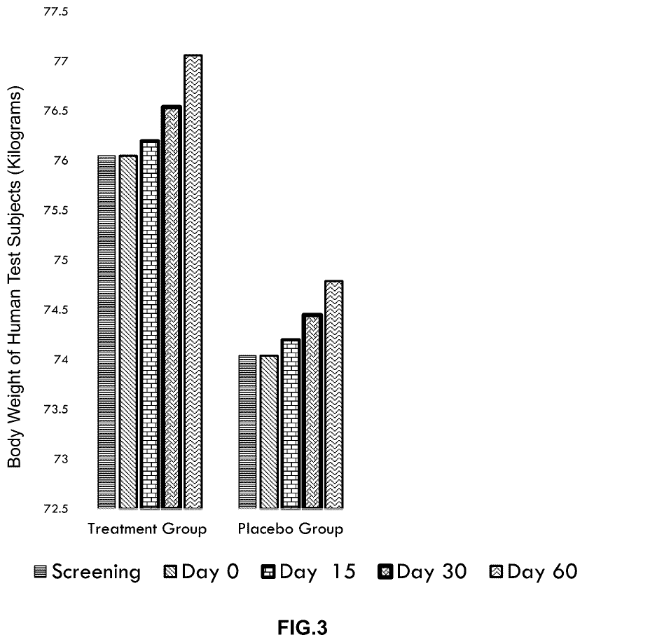

In accordance with the present disclosure, FIG. 3 illustrates a clustered column graph that describes the body weight distribution of the human beings who participated in the placebo-controlled human clinical trial. In FIG. 3, the body weight distribution is illustrated for a time period of sixty days, which is the total duration of the human clinical trial. In FIG. 3, the body weight distribution of the human participants is plotted on the vertical axis (y-axis), and the days on which the body weight-related readings were taken are indicated on the horizontal axis (x-axis). In accordance with the present disclosure, FIG. 3 is a graphical representation of the body weight-related readings illustrated in Table 5.

In accordance with the present disclosure, Table 6, provided below, illustrates the body mass index (BMI) of each of the subjects in both the treatment group and placebo group during the time period (sixty days) when the clinical trial was conducted.

TABLE 6
Body Mass Index (BMI) measurements of the clinical trial subjects

| | Body Mass Index- | |
|---|---|---|
| Values | BMI (Kilograms per meter square) | |
| represented as | Herbal Composition | |
| ‘mean ± SD’ | (Treatment Group) | Placebo Group |
| Screening | 25.78 ± 4.25 | 26.52 ± 4.27 |
| Day 0 | 25.96 ± 4.20 | 25.74 ± 2.57 |
| Day 15 | 26.02 ± 4.18 | 25.79 ± 2.58 |
| Day 30 | 26.13 ± 4.16 | 25.88 ± 2.56 |
| Day 60 | 26.31 ± 4.12 | 26.00 ± 2.54 |

In accordance with the present disclosure, Table 6 tabulates the body mass Index (BMI) variations across a time period of sixty days in each of the subjects who participated in the clinical trial. All the values in Table 6 are represented using the expression ‘mean±SD’, with the sample size (n) considered equivalent to twenty-four (i.e., sample size derived based on the total number of participants in the treatment group and placebo group).

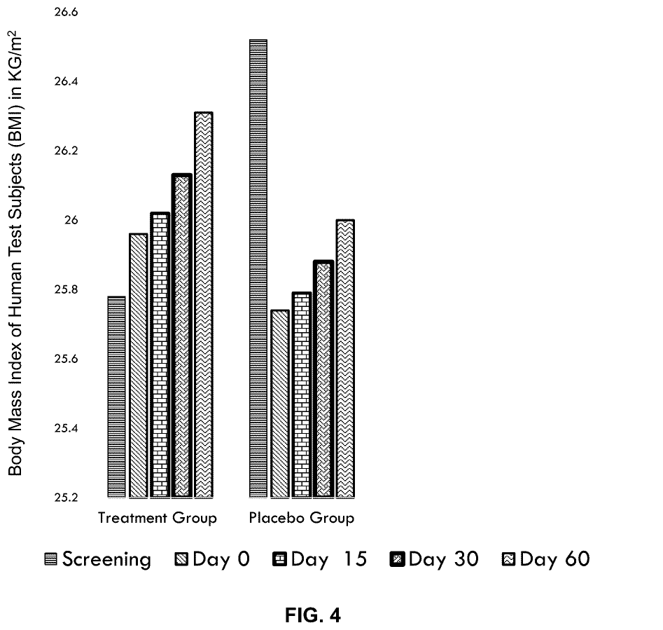

In accordance with the present disclosure, FIG. 4 illustrates a clustered column graph that describes the distribution of the body mass index (BMI) of the human beings who participated in the placebo-controlled human clinical trial. In FIG. 4, the body mass index distribution is illustrated for a time period of sixty days, which is the total duration of the human clinical trial. In FIG. 3, the body mass index (BMI) distribution of the human participants is plotted on the vertical axis (y-axis), and the days on which the BMI-related readings were taken are indicated on the horizontal axis (x-axis). In accordance with the present disclosure, FIG. 3 is a graphical representation of the body weight-related readings illustrated in Table 6.

From the values tabulated in Table 6, it is apparent that the herbal composition did not cause any abnormal or rapid variations in the BMI of the subjects who were administered the herbal composition daily for a period of sixty days and that the BMI of the subjects showed an extremely low, insignificant variation for the time period of sixty days during which the herbal composition was administered to them on a daily basis.

Therefore, from Table 6, it can be inferred that the herbal composition envisaged by the present disclosure does not cause any adverse changes in the BMI of the consumers/recipients when administered over a time period of at least sixty days, as indicated in the placebo-controlled human clinical study.

In accordance with the present disclosure, various vital parameters including temperature, heart rate, respiratory rate, and blood pressure (systolic and diastolic) of the subjects were recorded for a period of sixty days as a part of the clinical trial.

In accordance with the present disclosure, Table 7, provided below, illustrates the systolic blood pressure of each of the participants for a time period of sixty days.

TABLE 7
Systolic Blood Pressure measurements of the clinical trial subjects

| All the values | Systolic Blood Pressure (mm/Hg) | |
|---|---|---|
| represented as | Herbal Composition | |
| ‘mean ± SD’ | (Treatment Group) | Placebo Group |
| Screening | 121.54 ± 8.54 | 123.38 ± 5.43 |
| Day 0 | 118.46 ± 4.61 | 122.29 ± 5.02 |
| Day 15 | 118.96 ± 4.18 | 122.04 ± 5.06 |
| Day 30 | 118.38 ± 3.31 | 120.54 ± 3.80 |
| Day 60 | 120.54 ± 3.02 | 120.38 ± 4.33 |
| Change on | 2.08 ± 5.47 | −1.92 ± 5.15 |
| Day 60 | P-value = 0.0590 | P-value = 0.0910 |

In accordance with the present disclosure, Table 7 tabulates the systolic blood pressure variations across a time period of sixty days in each of the subjects who participated in the clinical trial. All the values in Table 7 are represented using the expression ‘mean±SD’, with the sample size (n) considered equivalent to twenty-four (i.e., sample size derived based on the total number of participants in the treatment group and placebo group).

From the values tabulated in Table 7, it is apparent that the herbal composition did not cause any abnormal or rapid variations in the systolic blood pressure of the subjects who were administered the herbal composition daily for a period of sixty days. Further, from Table 7, it can be inferred that the systolic blood pressure of the subjects showed extremely low and tolerable variations (i.e., variations between 118 mm/Hg and 121 mm/Hg, which can be construed as minor) throughout the time period of 60 days.

Further, the p-value obtained for systolic blood pressure change on ‘day 60’ for the treatment group is equivalent to 0.05, which is the typical, well-known threshold value for statistically determining the significance of systolic blood pressure readings. Typically, a p-value of 0.05 implies that there is only a five-percent chance that the observed readings were due to random variation. Therefore, a p-value that is less than or equal to 0.05 is used as the basis to reject the null hypothesis, which prescribes that any observed differences in data are due to a random chance rather than a specific cause. Therefore, with the p-value being equivalent to 0.05 for the change in systolic blood pressure obtained on ‘day 60’, it can be inferred that the systolic blood pressure readings provided in Table 7 are reasonably accurate and that the herbal composition envisaged by the present disclosure is unlikely to cause any adverse changes to the systolic blood pressure of the consumers/recipients when consumed over a time period of at least sixty days.

Further, Table 8, provided hereinbelow, illustrates the diastolic blood pressure of each of the participants of the clinical trial for a time period of sixty days.

TABLE 8
Diastolic Blood Pressure measurements of the clinical trial subjects

| All the values | Diastolic Blood Pressure (mm/Hg) | |
|---|---|---|
| represented as | Herbal Composition | |
| ‘mean ± SD’ | (Treatment Group) | Placebo Group |
| Screening | 76.21 ± 3.67 | 79.13 ± 6.14 |
| Day 0 | 76.42 ± 3.40 | 77.08 ± 4.75 |
| Day 15 | 79.33 ± 3.46 | 78.92 ± 3.87 |
| Change on | 2.92 ± 3.50 | 1.83 ± 6.21 |
| Day 15 | P-value = 0.0005 | P-value = 0.0846 |
| Day 30 | 79.42 ± 3.30 | 78.79 ± 3.44 |
| Change on | 3.00 ± 3.46 | 1.71 ± 6.29 |
| Day 30 | P-value = 0.0003 | P-value = 0.0466 |
| Day 60 | 79.08 ± 2.80 | 78.33 ± 2.57 |
| Change on | 2.67 ± 4.03 | 1.25 ± 5.33 |
| Day 60 | P-value = 0.0036 | P-value = 0.0551 |

In accordance with the present disclosure, Table 8 illustrates the diastolic blood pressure variations across a time period of sixty days in each of the subjects who participated in the clinical trial. All the values in Table 8 are represented using the expression ‘mean±SD’, with the sample size (n) considered equivalent to twenty-four (i.e., sample size derived based on the total number of participants in the treatment group and placebo group).

From the values tabulated in Table 8, it is apparent that the herbal composition did not cause any abnormal or rapid variations in the diastolic blood pressure of the subjects who were administered the herbal composition daily for a period of sixty days. Further, from Table 8, it can be inferred that the diastolic blood pressure of the subjects showed extremely low and tolerable variations (i.e., variations between 76 mm/Hg and 79 mm/Hg that can be considered minor) throughout the time period of 60 days.

Further, as illustrated in Table 8, the p-value obtained for diastolic blood pressure changes on days 15, 30, and 60 for the treatment group is less than 0.05, which is the typical, well-known threshold value for statistically determining the significance of diastolic blood pressure readings. Therefore, with the p-value being less than 0.05 for the diastolic blood pressure change-related readings obtained on days 15, 30, and 60, it can be inferred that the diastolic blood pressure readings provided in Table 8 are reasonably accurate and that the herbal composition envisaged by the present disclosure is unlikely to cause any adverse change in the diastolic blood pressure of the consumers/recipients when consumed over a time period of at least sixty days.

As discussed hitherto, in accordance with the present disclosure, the subjects who were administered the herbal composition were made to undertake several resistance training exercises, including bench press, leg press, cable pull down, squat, and deadlift inter-alia. Further, the subjects' performance of the said exercises was analysed to determine whether the herbal composition administered to the respective subjects enhanced their efficacy in terms of the handgrip strength, MUAC, and ability to perform the aforementioned exercises.

In accordance with the present disclosure, Table 9, provided below, illustrates the improvement in the grip strength of the subjects administered the herbal composition for a time period of sixty days. The grip strength-related values were recorded during days 0, 15, 30, and 60 of the clinical trial, and the improvement in grip strength of the subjects was established against predetermined baseline values on ‘day 60’ of the clinical trial. Preferably, the baseline values are the last ‘mean±SD’ values recorded with respect to the grip strength of each of the subjects in the treatment group and the placebo group before administering the herbal composition to the treatment group on ‘day 0’ of the clinical trial.

TABLE 9
Improvement in the grip strength of the subjects during the clinical trial.

| | Improvement in the Grip Strength of subjects | |
|---|---|---|
| All the values | (Kilograms) | |
| represented as | Herbal Composition | |
| ‘mean ± SD’ | (Treatment Group) | Placebo Group |
| Baseline | 45.81 ± 5.25 | 44.31 ± 3.35 |
| Day 15 | 47.03 ± 4.03 | 44.77 ± 3.28 |
| Day 30 | 50.10 ± 5.87 | 45.05 ± 1.94 |
| Day 60 | 50.33 ± 6.29 | 45.61 ± 2.37 |
| Change from the | 4.52 ± 1.85 | 1.3 ± 1.77 |
| Baseline value | | |

As illustrated in Table 9, the grip strength of the subjects belonging to the treatment group was enhanced significantly at the end of ‘day 60’ vis-à-vis the subjects of the placebo group. From Table 9, it is evident that while the grip strength of the subjects in the treatment group was enhanced by nearly four and a half kilograms against the baseline value over a period of sixty days, the subjects in the placebo group witnessed an improvement of only one and a half kilograms (approximately) over the baseline value during the same time period. Further, from Table 9, it is also apparent that the grip strength of the subjects in the treatment group was enhanced by about eleven percent over the baseline value in sixty days, while the grip strength of the subjects in the placebo group was enhanced only by two percent over the baseline value.

Therefore, the readings illustrated in Table 9 affirm that the herbal composition envisaged by the present disclosure, when administered for at least sixty consecutive days as a capsule of five hundred milligrams, enhances the grip strength of the consumers/recipients.

Likewise, Table 10, provided below, illustrates the improvements witnessed in the subjects belonging to the treatment group in terms of performing bench press exercise. From Table 10, it can be inferred that the subjects who were administered the herbal composition for a time period of sixty days were able to bench press with much heavier weights in comparison to the subjects of the placebo group. The bench press performance-related values were recorded during days 0, 15, 30, and 60 of the clinical trial, and the improvement in performing the bench press exercise was established on ‘day 60’ of the clinical trial against predetermined baseline values that were the last ‘mean±SD’ values recorded for each of the subjects in the treatment group and the placebo group for the bench press exercise, before administering the herbal composition to the treatment group on ‘day 0’ of the clinical trial.

TABLE 10
Improvement in the performance of bench press
exercise by the subjects during the clinical trial.

| | Improvement in performing bench | |
|---|---|---|
| All the values | press exercise (Kilograms) | |
| represented as | Herbal Composition | |
| ‘mean ± SD’ | (Treatment Group) | Placebo Group |
| Baseline | 41.67 ± 5.84 | 41.67 ± 5.45 |
| Day 15 | 44.42 ± 6.42 | 42.71 ± 4.75 |
| Day 30 | 47.63 ± 6.74 | 43.96 ± 4.53 |
| Day 60 | 50.96 ± 7.9 | 46.21 ± 4.29 |
| Change from the | 9.29 ± 7.87 | 4.4 ± 3.05 |
| Baseline value | | |

As illustrated in Table 10, subjects who were administered the herbal composition for sixty consecutive days were able to bench press with weights that were ten kilograms more in comparison to the baseline value, while the subjects in the placebo group were able to bench press with weights that were only four and a half kilograms more than the baseline value.

Further, as illustrated in Table 10, the subjects in the treatment group witnessed an improvement of about ten kilograms over the baseline value in terms of performing the bench press exercise over the time period of sixty days, and the subjects in the placebo group witnessed an improvement of approximately four and a half kilograms over the baseline value in terms of performing the same exercise over the same time period.

Further, as illustrated in Table 10, the ability of the subjects in the treatment group to perform the bench press exercise was enhanced by nearly twenty-two percent over the baseline value during the time period of sixty days (which is the total duration of the clinical trial), whereas the ability of the subjects in the placebo group to perform the bench press exercise was enhanced only by nearly twelve percent over the baseline value during the same time period.

Therefore, the readings illustrated in Table 10 affirm that the herbal composition envisaged by the present disclosure, when administered for at least sixty consecutive days as a capsule of five hundred milligrams, enhances the bench-pressing ability of the consumers/recipients.

Likewise, Table 11, provided below, illustrates the improvements witnessed in the subjects in terms of performing the leg press exercise. From Table 11, it can be inferred that the subjects who were administered the herbal composition for a time period of sixty days were able to perform the leg press exercise with much heavier weights in comparison to the subjects of the placebo group. The leg press performance-related values were recorded during days 0, 15, 30, and 60 of the clinical trial, and the improvement in performing the leg press exercise was established on ‘day 60’ of the clinical trial against predetermined baseline values that were the last ‘mean±SD’ values recorded for each of the subjects in the treatment group and the placebo group for the leg press exercise, before administering the herbal composition to the treatment group on ‘day 0’ of the clinical trial.

TABLE 11
Improvement in the performance of leg press
exercise by the subjects during the clinical trial.

| | Improvement in performing leg | |
|---|---|---|
| All the values | press exercise (Kilograms) | |
| represented as | Herbal Composition | |
| ‘mean ± SD’ | (Treatment Group) | Placebo Group |
| Baseline | 174.58 ± 44.23 | 174.79 ± 14.1 |
| Day 15 | 185.00 ± 36.54 | 177.50 ± 14.45 |
| Day 30 | 190.42 ± 37.62 | 180.63 ± 14.99 |
| Day 60 | 200.00 ± 39.34 | 186.67 ± 15.08 |
| Change from the | 25.42 ± 3.27 | 11.88 ± 9.42 |
| Baseline value | | |

Further, as illustrated in Table 11, the subjects in the treatment group witnessed an improvement of about twenty-five kilograms over the baseline value in terms of performing the bench press exercise over the time period of sixty days, and the subjects in the placebo group witnessed an improvement of only approximately four and a half kilograms over the baseline value in terms of performing the bench press exercise over the same time period.

Further, as illustrated in Table 11, the subjects in the treatment group witnessed an improvement of about fourteen percent over the baseline value in terms of performing the leg press exercise over the duration of sixty days, and the subjects in the placebo group witnessed an improvement of only seven percent in terms of performing the same exercise over the same time duration.

Therefore, the readings illustrated in Table 11 affirm that the herbal composition envisaged by the present disclosure, when administered for at least sixty consecutive days as a capsule of five hundred milligrams, enhances the ability of the consumers/recipients to perform leg press exercises.

Likewise, Table 12, provided below, illustrates the improvements witnessed in the subjects in terms of performing the cable pulldown exercise. From Table 12, it can be inferred that the subjects who were administered the herbal composition for a time period of sixty days were able to perform the cable pulldown exercise with much heavier weights in comparison to the subjects of the placebo group.

In accordance with the present disclosure, the cable pulldown performance-related values were recorded during days 0, 15, 30, and 60 of the clinical trial, and the improvement in performing the cable pulldown exercise was established on ‘day 60’ of the clinical trial against predetermined baseline values.

In accordance with the present disclosure, the predetermined base values for the treatment group and placebo group are the last ‘mean±SD’ values recorded for each of the subjects in the respective groups in relation to the performance of the cable pulldown exercise, before the herbal composition was administered to the treatment group on ‘day 0’ of the clinical trial.

TABLE 12
Improvement in the performance of cable pulldown
exercise by the subjects during the clinical trial.

| | Improvement in performing cable | |
|---|---|---|
| All the values | pulldown exercise (Kilograms) | |
| represented as | Herbal Composition | |
| ‘mean ± SD’ | (Treatment Group) | Placebo Group |
| Baseline | 79.17 ± 18.33 | 79.63 ± 16.92 |
| Day 15 | 88.89 ± 19.1 | 83.39 ± 15.76 |
| Day 30 | 94.93 ± 20.71 | 84.39 ± 12.79 |
| Day 60 | 105.56 ± 21.98 | 90.28 ± 15.48 |
| Change from the | 26.39 ± 14.21* | 10.65 ± 12.47 |
| Baseline value | | |

As illustrated in Table 12, the subjects in the treatment group witnessed an improvement of about twenty-six kilograms over the baseline value in terms of performing the cable pulldown exercise over the time period of sixty days, and the subjects in the placebo group witnessed an improvement of only ten kilograms over the baseline value in terms of performing the same exercise over the same time period.

Further, as illustrated in Table 12, the subjects in the treatment group witnessed an improvement of about thirty-three percent over the baseline value in terms of performing the cable pulldown exercise over the time period of sixty days, while the subjects in the placebo group witnessed an improvement of only fourteen percent over the baseline value in terms of performing the same exercise over the same time period.

Therefore, the readings illustrated in Table 12 affirm that the herbal composition envisaged by the present disclosure, when administered for at least sixty consecutive days as a capsule of five hundred milligrams, enhances the ability of the consumers/recipients to perform cable pulldown exercises.

Likewise, the mid-upper arm circumference (MUAC) of the subjects who were administered the herbal composition for sixty consecutive days was enhanced by about approximately two centimeters (cm) over the baseline value, while the subjects in the placebo group witnessed an enhancement of only one centimeter (cm) over the baseline value. In this case, the subjects of the treatment group witnessed an improvement of six percent over the baseline value over a time period of sixty days, while the subjects of the placebo group witnessed an improvement of three percent over the baseline value for the same time period.

Therefore, it is apparent that the herbal composition, when administered for at least sixty consecutive days as a capsule of five hundred milligrams, enhances the mid-upper arm circumference of the consumers/recipients, thereby improving their muscle strength.

Likewise, on ‘day 60’, the subjects who were administered the herbal composition for sixty consecutive days were able to run on a treadmill for about seventeen minutes at a speed of six kilometers per hour and zero inclination, with the inclination of the treadmill being increased by two percent every two minutes. On the contrary, the subjects in the placebo group were able to run for only fifteen minutes on the same day (i.e., day 60) with the same treadmill settings.

Further, while the subjects who were administered the herbal composition witnessed an improvement of about five minutes over the baseline value in terms of their treadmill time on ‘day 60’ with the treadmill settings described above, the subjects in the placebo group witnessed an improvement of only approximately two minutes over the baseline value on the same day (i.e., day 60) and with the same treadmill settings.

In this case, the subjects of the treatment group witnessed an improvement of forty-five percent over the baseline value over a time period of sixty days, while the subjects of the placebo group witnessed an improvement of seventeen percent over the baseline value for the same time period for the same exercise (i.e., running on the treadmill).

Therefore, it is affirmed that the herbal composition, when administered for at least sixty consecutive days as a capsule of five hundred milligrams, enhances the recipients' ability to perform physically demanding cardiovascular exercises by enhancing their endurance.

In accordance with the present disclosure, the results achieved by the clinical trial also demonstrated a marked increase in the total Testosterone levels of the subjects (categorized as the treatment group) that were administered the herbal composition in a dosage of five hundred milligrams for the time period of sixty consecutive days, in comparison to the total Testosterone levels of the subjects who were a part of the placebo group and did not receive any dosage of the herbal composition.

In accordance with the present disclosure, Table 13, provided below, illustrates the total Testosterone levels observed in the subjects that were administered the herbal composition for sixty consecutive days. In accordance with the present disclosure, Table 13 indicates the total Testosterone levels observed on days 15, 30, and 60 of the clinical trial in the treatment group as well as the placebo group.

TABLE 13
Total Testosterone Levels observed in subjects administered
with the herbal composition during the clinical trial.

| | Improvement in Total Testosterone Levels | |
|---|---|---|
| All the values | (ng/dL) | |
| represented as | Herbal Composition | |
| ‘mean ± SD’ | (Treatment Group) | Placebo Group |
| Baseline | 401.35 ± 21.57 | 398.51 ± 30.91 |
| Day 15 | 442.80 ± 23.00 | 402.46 ± 30.74 |
| Day 30 | 478.28 ± 24.88 | 412.38 ± 30.69 |
| Day 60 | 540.54 ± 24.13 | 446.21 ± 31.28 |
| Change from the | 139.19 ± 9.55 | 47.70 ± 2.10 |
| Baseline value | (p-value < 0.0001) | (p-value < 0.0001) |

In accordance with the present disclosure, the total Testosterone level-related values described in Table 13 for days 15, 30, and 60 were calculated using the expression ‘mean±SD,’ with the sample size (n) being equivalent to twenty-four (i.e., sample size derived based on the total number of participants in each of the treatment group and placebo group). Typically, the expression ‘mean±standard deviation (mean±SD)’ depicts the central value (the mean) along with a measure of the spread or variability (the standard deviation).

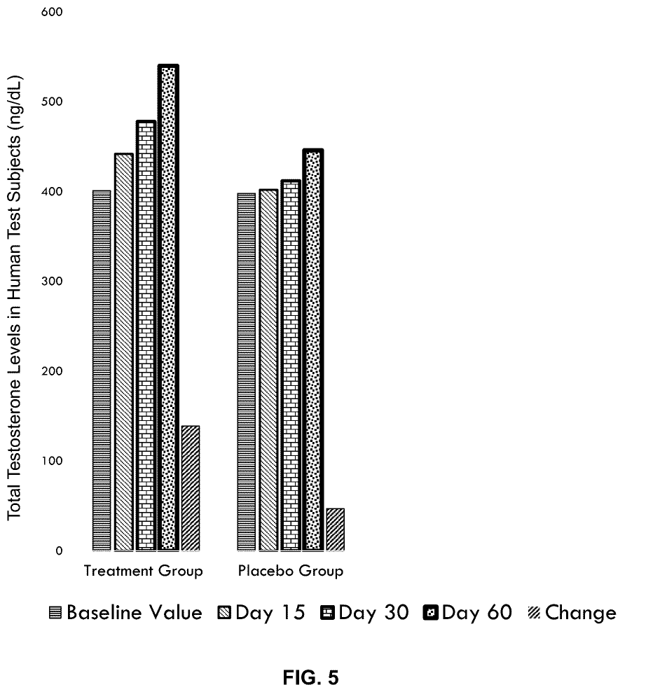

In accordance with the present disclosure, FIG. 5 illustrates a clustered column graph that describes the increase in the total Testosterone levels of the human beings who participated in the placebo-controlled human clinical trial. In FIG. 5, the increase in the total Testosterone levels is illustrated for a time period of sixty days, which is the total duration of the human clinical trial. In FIG. 5, the increase in the total Testosterone levels is plotted on the vertical axis (y-axis), and the days on which the total Testosterone level-related readings were taken are indicated on the horizontal axis (x-axis). In accordance with the present disclosure, FIG. 5 is a graphical representation of the body weight-related readings illustrated in Table 13.

Further, the values of ‘mean±SD’ corresponding to the treatment group were statistically compared with the ‘mean±SD’ values corresponding to the placebo group for days 15, 30, and 60. It was observed that the ‘mean±SD’ values corresponding to the treatment group showed a significant increase in terms of total Testosterone levels, vis-à-vis the values corresponding to the placebo group.

In any statistical analysis, the p-value suggests strong evidence against the null hypothesis and provides for the null hypothesis to be rejected in favor of the alternative hypothesis. Further, when the efficacy and efficiency of the herbal composition of the present disclosure is tested through the clinical trial, the ‘null hypothesis’ is that the “herbal composition has no effect.”

As illustrated in Table 13, the p-value obtained at the end of ‘day 60’ for the total Testosterone levels of the subjects belonging to the treatment group is less than 0.0001, a value significantly lesser than the typical, well-known threshold value of 0.05. Typically, a p-value less than the threshold value 0.05 implies less than a five-percent chance that the readings (described in Table 13) occurred due to a random variation, thus satisfying the null hypothesis.

Further, since the p-value for the total Testosterone levels of the subjects belonging to the treatment group is remarkably lesser than the threshold value of 0.05, the total Testosterone readings/values depicted in Table 13 demonstrate the effectiveness and efficacy of the herbal composition in terms of enhancing the total Testosterone levels in human beings and thus form the basis for the rejection of the null hypothesis, and indicate that the possibility of the null hypothesis being true, in this case, would not exceed five percent.

Preferably, the baseline values illustrated in Table 13 are the last ‘mean±SD’ values recorded for each of the subjects in both the treatment group and placebo group on ‘day 0’ of the clinical trial, the day on which the subjects of the treatment group are administered with the first dose of the herbal composition. Further, as illustrated in Table 13, the total Testosterone levels of the subjects in the treatment group were enhanced by one hundred and thirty-nine decilitres per nanogram over the baseline value, while the total Testosterone levels of the subjects in the placebo group increased only by forty-eight decilitres per nanogram over the baseline value.

Further, as per the values provided in Table 13, the total Testosterone levels of the subjects in the treatment group were enhanced by thirty-five percent over the baseline value during the time period of sixty days, while the total Testosterone levels of the subjects in the placebo group were enhanced only by twelve percent over the same time period.

Therefore, based on the values illustrated in Table 13, it is apparent that the herbal composition, when administered for at least sixty consecutive days as a capsule of five hundred milligrams, enhances the total Testosterone levels of the recipients (human beings, especially men) by at least thirty-five percent in comparison to the baseline value, and thus marks a significant improvement over the total Testosterone levels observed in the placebo group that did not receive the herbal composition.

In accordance with the present disclosure, the results achieved by the clinical trials also demonstrated a marked increase in the free Testosterone levels of the subjects (categorized as the treatment group) that were administered the herbal composition in a dosage of five hundred milligrams for the time period of sixty consecutive days, in comparison to the free Testosterone levels of the subjects who were a part of the placebo group and did not receive any dosage of the herbal composition.

In accordance with the present disclosure, Table 14, provided below, illustrates the free Testosterone levels observed in the subjects that were administered the herbal composition for sixty consecutive days. In accordance with the present disclosure, Table 14 indicates the free Testosterone levels observed on days 15, 30, and 60 of the clinical trial in the treatment group as well as the placebo group.

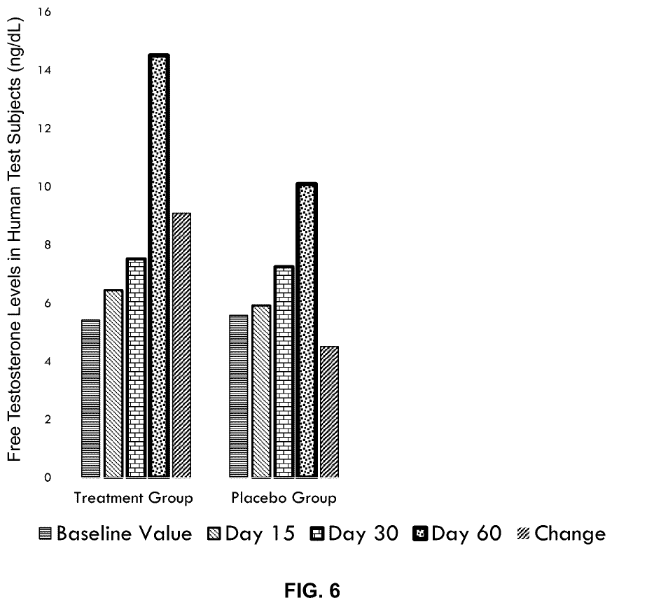

In accordance with the present disclosure, FIG. 6 illustrates a clustered column graph that describes the increase in the free Testosterone levels of the human beings who participated in the placebo-controlled human clinical trial. In FIG. 6, the increase in the free Testosterone levels is illustrated for a time period of sixty days, which is the total duration of the human clinical trial.

In FIG. 6, the increase in the free Testosterone levels is plotted on the vertical axis (y-axis), and the days on which the free Testosterone level-related readings were taken are indicated on the horizontal axis (x-axis). In accordance with the present disclosure, FIG. 6 is a graphical representation of the body weight-related readings illustrated in Table 14.

In accordance with the present disclosure, the free Testosterone level-related values described in Table 14 for days 15, 30, and 60 were calculated using the expression ‘mean±SD,’ with the sample size (n) being equivalent to twenty-four (i.e., sample size derived based on the total number of participants in the treatment group and placebo group).

TABLE 14
Free Testosterone Levels observed in subjects administered
with the herbal composition during the clinical trial.

| | Improvement in Free Testosterone Levels | |
|---|---|---|
| All the values | (ng/dL) | |
| represented as | Herbal Composition | |
| ‘mean ± SD’ | (Treatment Group) | Placebo Group |
| Baseline | 5.42 ± 0.41 | 5.58 ± 0.42 |
| Day 15 | 6.44 ± 0.58 | 5.92 ± 0.40 |
| Day 30 | 7.52 ± 0.94 | 7.25 ± 0.47 |
| Day 60 | 14.51 ± 2.21 | 10.09 ± 0.88 |
| Change from the | 9.09 ± 2.11 | 4.51 ± 0.98 |
| Baseline value | (p-value < 0.0001) | (p-value < 0.0001) |

Further, the values of ‘mean±SD’ of the treatment group were statistically compared with the ‘mean±SD’ values of the placebo group for days 15, 30, and 60. It was observed that the ‘mean±SD’ values corresponding to the treatment group showed a reasonable increase in terms of free Testosterone levels vis-à-vis the values corresponding to the placebo group.

As illustrated in Table 14, the p-value obtained at the end of day 60 for free Testosterone levels of the subjects belonging to the treatment group is less than 0.0001, a value significantly lesser than the typical, well-known threshold value of 0.05. Further, since the p-values for free Testosterone levels of the subjects belonging to the treatment group are remarkably lesser than the threshold value of 0.05, the total Testosterone readings/values depicted in Table 14 demonstrate the effectiveness and efficacy of the herbal composition envisaged by the present disclosure in enhancing the free Testosterone levels of the recipients (i.e., human beings).

Further, as per the values described in Table 14, the free Testosterone levels of the subjects in the treatment group were enhanced by nine decilitres per nanogram over the baseline value, while the total Testosterone levels of the subjects in the placebo group increased only by four and a half decilitres per nanogram over the baseline value.

Further, as per the values provided in Table 14, the free Testosterone levels of the subjects in the treatment group were enhanced by hundred and eighty percent over the baseline value during the time period of sixty days, while the free Testosterone levels of the subjects in the placebo group were enhanced only by hundred percent over the same time period.

Therefore, based on the values illustrated in Table 14, it is apparent that the herbal composition, when administered for at least sixty consecutive days as a capsule of five hundred milligrams, enhances the free Testosterone levels of the recipients (human beings, especially men) by at least hundred and eighty percent in comparison to the baseline value, and thus marks a significant improvement over the free Testosterone levels observed in the placebo group that did not receive the herbal composition.

In accordance with the present disclosure, the herbal composition, when administered in a dosage of five hundred milligrams for the time period of sixty consecutive days, also elevates the mechanistic target of rapamycin (mTOR) levels. In accordance with the present disclosure, the results achieved by the clinical trials also demonstrated a marked increase in the mTOR levels of the subjects (categorized as the treatment group) that were administered the herbal composition in the above-mentioned dosage for the above-mentioned time period, in comparison to the mTOR levels of the subjects who were a part of the placebo group and did not receive any dosage of the herbal composition.

In accordance with the present disclosure, Table 15, provided below, illustrates the mTOR levels observed in the subjects that were administered the herbal composition for sixty consecutive days. In accordance with the present disclosure, Table 15 indicates the mTOR levels observed on day 30 and day 60 of the clinical trial in the treatment group as well as the placebo group.

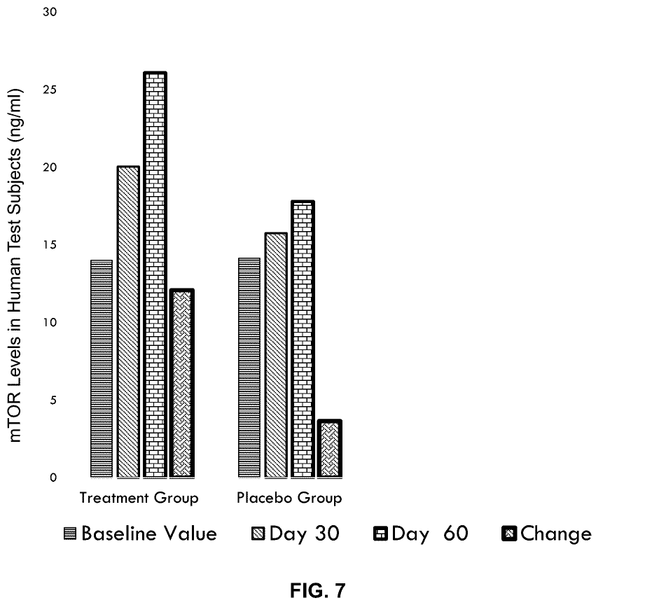

In accordance with the present disclosure, FIG. 7 illustrates a clustered column graph that describes the increase in the mTOR levels of the human beings who participated in the placebo-controlled human clinical trial. In FIG. 7, the increase in the mTOR levels is illustrated for a time period of sixty days, which is the total duration of the human clinical trial. In FIG. 7, the increase in the mTOR levels is plotted on the vertical axis (y-axis), and the days on which the mTOR level-related readings were taken are indicated on the horizontal axis (x-axis). In accordance with the present disclosure, FIG. 7 is a graphical representation of the body weight-related readings illustrated in Table 15.

TABLE 15
mTOR Levels observed in subjects administered with
the herbal composition during the clinical trial.

| All the values | Improvement in mTOR Levels (ng/ml) | |
|---|---|---|
| represented as | Herbal Composition | |
| ‘mean ± SD’ | (Treatment Group) | Placebo Group |
| Baseline | 14.01 ± 2.33 | 14.15 ± 3.45 |
| Day 30 | 20.05 ± 3.52 | 15.76 ± 3.70 |
| Day 60 | 26.09 ± 4.52 | 17.80 ± 3.61 |
| Change from the | 12.08 ± 4.79 | 3.65 ± 0.84 |
| Baseline value | (p-value < 0.0001) | (p-value < 0.0001) |

In accordance with the present disclosure, the mTOR level-related values described in Table 15 for days 30 and 60 of the clinical trial were calculated using the expression ‘mean±SD,’ with the sample size (n) being equivalent to twenty-four (i.e., sample size derived based on the total number of participants in the treatment group and placebo group).

Further, the values of ‘mean±SD’ of the treatment group were statistically compared with the ‘mean±SD’ values of the placebo group for days 30 and 60. It was observed that the ‘mean±SD’ values corresponding to the treatment group showed a reasonable increase in terms of mTOR levels vis-à-vis the ‘mean±SD’ values corresponding to the placebo group.

As per Table 15, the p-value obtained on day 60 for mTOR levels of the subjects belonging to the treatment group is less than 0.0001, a value significantly lesser than the typical, well-known threshold value of 0.05. Further, since the p-value for mTOR levels of the subjects belonging to the treatment group is remarkably lesser than the threshold value of 0.05, the mTOR level-related values depicted in Table 15 demonstrate the effectiveness and efficacy of the herbal composition envisaged by the present disclosure in enhancing the mTOR levels of the recipients.

Further, as per the values described in Table 15, the mTOR levels of the subjects in the treatment group were enhanced by twelve decilitres per milligram over the baseline value, while the mTOR levels of the subjects in the placebo group increased only by approximately three and a half decilitres per milligram over the baseline value.

Further, as per the values provided in Table 15, the mTOR levels of the subjects in the treatment group were enhanced by eighty-six percent over the baseline value during the time period of sixty days, while the mTOR levels of the subjects in the placebo group were enhanced only by twenty-two percent over the same time period.

Therefore, based on the values illustrated in Table 15, it is apparent that the herbal composition, when administered for at least sixty consecutive days as a capsule of five hundred milligrams, enhances the mTOR levels of the recipients (human beings, especially men) by at least eighty-five percent in comparison to the baseline value, and thus marks a significant improvement over the mTOR levels observed in the placebo group that did not receive the herbal composition.

In accordance with the present disclosure, the herbal composition, when administered in a dosage of five hundred milligrams for the time period of sixty consecutive days, also reduces Lactate Dehydrogenase (LDH) levels in human beings. LDH is typically found in various tissues. Typically, elevated levels of LDH in the blood often indicate cell damage or tissue injury. Further, when cells are damaged, LDH is released into the bloodstream. Elevated levels of LDH often can be indicative of a heart attack or other forms of heart damage, liver damage, muscle damage, cancer, haemolysis (destruction of red blood cells), renal disease, pneumonia, and sepsis.

In accordance with the present disclosure, Table 16, provided below, illustrates the LDH levels observed in the subjects that were administered the herbal composition for sixty consecutive days. In accordance with the present disclosure, Table 16 indicates the mTOR levels observed on days 30 and 60 of the clinical trial in the treatment group as well as the placebo group.

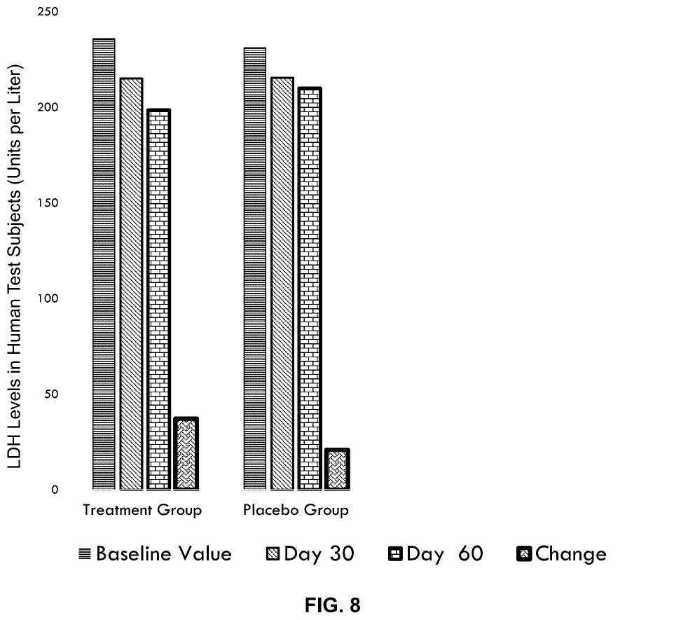

In accordance with the present disclosure, FIG. 8 illustrates a clustered column graph that describes the regulation and preferably the decrease in the mTOR levels of the human beings who participated in the placebo-controlled human clinical trial. In FIG. 8, the LDH levels are illustrated for a time period of sixty days, which is the total duration of the human clinical trial. In FIG. 8, the LDH levels are plotted on the vertical axis (y-axis), and the days on which the LDH level-related readings were taken are indicated on the horizontal axis (x-axis). In accordance with the present disclosure, FIG. 8 is a graphical representation of the body weight-related readings illustrated in Table 16.

TABLE 16
LDH Levels observed in subjects administered with
the herbal composition during the clinical trial.

| All the values | LDH Levels (Units per Liter) | |
|---|---|---|
| represented as | Herbal Composition | |
| ‘mean ± SD’ | (Treatment Group) | Placebo Group |
| Baseline | 235.92 ± 20.24 | 231.05 ± 15.23 |
| Day 30 | 215.08 ± 20.59 | 215.44 ± 17.23 |
| Day 60 | 198.67 ± 19.28 | 210.06 ± 13.15 |
| Change from the | (37.25) ± 5.09 | (20.99) ± 4.89 |
| Baseline value | (p-value < 0.0001) | (p-value < 0.0001) |

In accordance with the present disclosure, the LDH level-related values described in Table 16 for days 30 and 60 were calculated using the expression ‘mean±SD,’ with the sample size (n) being equivalent to twenty-four (i.e., sample size derived based on the total number of participants in the treatment group and placebo group).

Further, the values of ‘mean±SD’ of the treatment group were statistically compared with the ‘mean±SD’ values of the placebo group for days 30 and 60. It was observed that the ‘mean±SD’ values corresponding to the treatment group showed a reasonable decrease in terms of the LDH levels vis-à-vis the ‘mean±SD’ values corresponding to the placebo group.

As per Table 16, the p-value obtained on day 60 for LDH levels of the subjects belonging to the treatment group is less than 0.0001, a value significantly lesser than the typical, well-known threshold value of 0.05. Further, since the p-value for LDH levels of the subjects belonging to the treatment group is remarkably lesser than the threshold value of 0.05, the LDH level-related values depicted in Table 16 demonstrate the effectiveness and efficacy of the herbal composition envisaged by the present disclosure, in terms of regulating the LDH levels in human beings.

Further, as per the values described in Table 16, the LDH levels of the subjects in the treatment group were decreased by thirty-seven units per Liter over the baseline value, while the LDH levels of the subjects in the placebo group decreased only by approximately twenty units per Liter over the baseline value.

Therefore, based on the values illustrated in Table 16, it is apparent that the herbal composition, when administered for at least sixty consecutive days as a capsule of five hundred milligrams, effectively regulates the LDH levels of the recipients by at least sixteen percent in comparison to the baseline value. On the contrary, the decrease in LDH levels witnessed in the placebo group is only nine percent.

Therefore, based on the values illustrated in Table 16, it is apparent that the herbal composition, when administered for at least sixty consecutive days as a capsule of five hundred milligrams, effectively regulates the LDH levels of the recipients (human beings, especially men).

From the values illustrated in tables 1-4 and the in-vivo efficacy study performed on the Spring Dawley rats, it is apparent that the herbal composition envisaged by the present disclosure brings about a remarkable and significant improvement in the total Testosterone levels, free Testosterone levels and sexual behavior of rats, and thus affirms the efficacy of the herbal composition in enhancing the Testosterone levels and sexual behavior of the rats.

From the values illustrated in tables 5-16 and the placebo-controlled clinical trial performed on human beings, it is affirmed that the herbal composition envisaged by the present disclosure: a) does not cause any abnormal variations in the body weight and BMI of the human recipients, b) does not cause any abnormal variations in the systolic blood pressure and diastolic blood pressure of the human recipients, c) significantly enhances the ability of the human recipients to perform a variety of resistance training exercises, d) improves the MUAC and time-to-exhaustion of the human recipients, e) significantly enhances the total Testosterone levels, free Testosterone levels, and mTOR levels in human recipients, and f) decreases the LDH levels in human recipients.

### Technical Advantages

The technical advantages envisaged by the present disclosure include the realization of a safe-to-use, side effects-free herbal composition that, when administered in a therapeutically active and appropriate manner, enhances the anabolic and androgenic activities in the human body, and manifests improved testosterone levels, improved and sustained energy levels, sustained vigor, enhanced stamina, healthy aging, cellular longevity, enhanced muscle mass, enhanced muscle strength, enhanced sexual functions, psychological well-being, and reduced stress levels.

Yet another technical advantage envisaged by the herbal composition of the present disclosure includes alleviation of symptoms associated with low levels of testosterone in the human body, including loss of libido, loss of erectile function, abdominal obesity, decreased muscle mass and strength, fatigue, mood swings, decreased bone density, decreased motivation, decreased memory, and concentration.

The herbal composition envisaged by the present disclosure caused a significant increase in free testosterone levels and total testosterone levels of humans (preferably men) when administered in a therapeutically active and appropriate quantity for a time period of at least sixty days.

Yet another technical advantage envisaged by the herbal composition of the present disclosure is that it is chemical-free and does not entail any harmful side effects typically associated with well-known Testosterone boosters such as nitric oxide and MSH.

Yet another technical advantage envisaged by the herbal composition of the present disclosure is that it does not exhibit any adverse behavioral patterns in people who consume the herbal composition, even for prolonged time periods. The herbal composition neither exhibits any adverse clinical symptoms nor brings about any abnormal behavioral patterns on the part of the consumer/recipient (human beings, preferably men). The herbal composition envisaged by the present disclosure also does not bring about any significant change in the body composition of the consumer, particularly an increase in the body weight of the consumer.

Further, the herbal composition brings about a significant increase in the Testosterone levels of the consumer while keeping all the biochemistry-related parameters of the human body, viz., urea, creatinine, uric acid, bilirubin, calcium, and liver function-related parameters, largely unchanged. Further, the herbal composition envisaged by the present disclosure exhibits a good safety profile and enables consumers (human beings, especially men) to perform muscle-building exercises with a better form and heavier weights and recover at a faster rate in comparison to the existing health supplements. Further, the herbal composition envisaged by the present disclosure exhibits a marked improvement in key serum biomarkers, including insulin, mTOR (mechanistic target of rapamycin), lactate, LDH (lactate dehydrogenase), CK (Creatine Kinase), in addition to enhancing the Testosterone levels in human beings, especially men.

What is claimed is:

1. A herbal composition suitable for enhancing sexual wellness and muscle strength in human beings, said herbal composition comprising:
an effective amount of an extract of Tamarindus indica and an effective amount of an extract of Moringa oleifera, and wherein the herbal composition comprises the extract of Tamarindus indica in an amount ranging between 10% to 90% by weight of said herbal composition and the extract of Moringa oleifera in an amount ranging between 10% to 90% by weight of said herbal composition;
at least one additional extract selected from a group consisting of Cocculus hirsutus extract in an amount up to 35% by weight of said herbal composition, Coffea arabica extract in an amount up to 10% by weight of said herbal composition, Psoralea corylifolia extract in an amount up to 60% by weight of said herbal composition, Cinnamon zeylanicum extract in an amount up to 35% by weight of said herbal composition, and Brassica nigra extract in an amount up to 10% by weight of said herbal composition.

2. The herbal composition as claimed in claim 1, wherein the extract of Tamarindus indica is standardized to contain polysaccharides in a concentration ranging between 0.1% and 60% by weight of said herbal composition, and to contain proanthocyanidins in a concentration ranging between 0.1% and 60% by weight of said herbal composition.

3. The herbal composition as claimed in claim 1, wherein the extract of Moringa oleifera is standardized to contain polysaccharides with a concentration in a range of 0.1% to 30% by weight of said herbal composition, and to contain saponins with a concentration in a range of 0.1% to 50% by weight of said herbal composition.

4. The herbal composition as claimed in claim 1, wherein said herbal composition comprises at least one of fractions, active compounds, phytochemicals, or mixtures thereof derived from plant parts of the Tamarindus indica, the plant parts of the Moringa oleifera, and the plant parts of at least one of Cocculus hirsutus, Coffea arabica, Psoralea corylifolia, Cinnamon zeylanicum and Brassica nigra.

5. The herbal composition as claimed in claim 4, wherein said plant parts are selected from a group consisting of leaves, stems, tender stems, tender twigs, aerial parts, whole fruits, fruit rinds, seeds, roots, bark, and hardwood.

6. The herbal composition as claimed in claim 1, wherein said herbal composition further comprises an extract of at least one herb selected from a group consisting of Allium cepa, Coffea robusta, Mimosa pudica, Abutilon indicum, Withania somnifera, Tribulus terrestris, and Coleus forskohlii, and wherein said extract of at least one herb is in an amount ranging between 10% and 40% by weight of said herbal composition.

7. The herbal composition as claimed in claim 1, wherein said herbal composition comprises at least one additional component selected from a group consisting of biological agents, pharmaceutically acceptable active ingredients, vitamins, minerals, excipients, and carriers and diluents, and wherein said additional component is in an amount up to 10% by weight of said herbal composition.

8. The herbal composition as claimed in claim 7, wherein said excipients are selected from a group consisting of monosaccharides, disaccharides, polycarbonates, and modified starch.

9. The herbal composition as claimed in claim 7, wherein said carriers and diluents are selected from a group consisting of polyhydric alcohols, sugar alcohols, cellulose-based derivatives, silicates, metallic stearates, organic acids, fatty acid esters, esters of polysorbate, Vitamin B, nicotinamide, calcium pantothenate, amino acids, proteins, and organic metal salts.

10. A method of enhancing sexual wellness and muscle strength in a person, the method comprising:
administering said person with a herbal composition that includes an effective amount of an extract of Tamarindus indica and an effective amount of an extract of Moringa oleifera, said herbal composition comprising:
the extract of Tamarindus indica in an amount ranging between 10% to 90% by weight of said herbal composition and the extract of Moringa oleifera in an amount ranging between 10% to 90% by weight of said herbal composition;
at least one additional extract selected from a group consisting of Cocculus hirsutus extract in an amount up to 35% by weight of said herbal composition, Coffea arabica extract in an amount up to 10% by weight of said herbal composition, Psoralea corylifolia extract in an amount up to 60% by weight of said herbal composition, Cinnamon zeylanicum extract in an amount up to 35% by weight of said herbal composition, and Brassica nigra extract in an amount up to 10% by weight of said herbal composition.

11. The method as claimed in claim 10, wherein the method further includes supplementing said person with a therapeutically effective dosage of said herbal composition.

12. The method as claimed in claim 10, wherein enhancing sexual wellness and muscle strength in said person further includes enhancing anabolic and androgenic activities in said person's body, which results in at least one health benefit selected from a group consisting of: improved testosterone levels, improved energy levels, sustained energy levels, sustained vigor, enhanced stamina, healthy aging, cellular longevity, enhanced muscle mass, enhanced muscle strength, enhanced sexual functions, psychological well-being, and reduced stress levels.

13. The method as claimed in claim 10, wherein enhancing sexual wellness and muscle strength in said person further includes alleviating at least one symptom associated with low levels of testosterone in said person's body, wherein said at least one symptom is selected from a group consisting of loss of libido, loss of erectile function, abdominal obesity, decreased muscle mass and strength, fatigue, mood swings, decreased bone density, decreased motivation, decreased memory and concentration.
